(19) United States
(12) Patent Application Publication
(10) Pub. No.: US 2005/0163771 A1
(43) Pub. Date: Jul. 28, 2005

(54) Method and pharmaceutical composition for inhibiting premature rapture of fetal membranes, ripening of uterine cervix and preterm labor in mammals

(76) Inventor: Shamir Leibovitz, Tel Aviv (IL)

Correspondence Address: Martin Moynihan;c/o Anthony Castorina, Suite 207, 2001 Jefferson Davis Highway, Arlington, VA, 22202, US

(21) Appl. No.: 11/080,474

(22) Filed: Mar. 16, 2005

Related U.S. Application Data
(63) Continuation of application No. 10/286,959, filed on Nov. 4, 2002.
Application No. 10/286,959 is a continuation of application No. 09/554,124, filed on May 9, 2000, now abandoned.

(30) Foreign Application Priority Data
Nov. 24, 1997 (IL) 122278

(51) Int. Cl. A61K 39/395; A61K 39/40; A61K 39/42

(52) U.S. Cl. 424/133.1

(57) ABSTRACT

A method and a pharmaceutical composition for inhibiting premature rapture of the fetal membranes, ripening of the uterine cervix and preterm labor of female mammals including human. The method includes the step of administering compounds for reversing at least two biochemical conditions being associated with the above processes. The pharmaceutical composition includes compounds for reversing at least two biochemical conditions being associated with the above processes.

This is a continuation of U.S. patent application No. 10/286,959, filed Nov. 4, 2002, which is a continuation of U.S. patent application No. 09/554,124, filed May 9, 2000, which is a National Phase Application of PCT/IL98/00572, filed Nov. 24, 1998, which claims the benefit of priority from Israeli Patent Application No. 122278, filed Nov. 24, 1997.

### FIELD AND BACKGROUND OF THE INVENTION

The present invention relates to a method and a pharmaceutical composition for preterm labor inhibition and, more particularly, to a method and a pharmaceutical composition for inhibiting premature rapture of the fetal membranes, ripening of the uterine cervix and, as a direct result, inhibiting preterm labor in mammals.

A number of factors, also referred to as biochemical conditions, take active part during gestation and labor. Preterm labor due to premature rapture of the fetal membranes and ripening of the cervix is, in many cases, a result of impaired balance among these factors, the important of which being the progesterone/estrogen ratio, prostaglandins, collagenase and matrix metalloproteinases, cytokines, oxytocin and nitric oxide.

In the following, the effect each of these factors has on the process of labor and membranes rapture and cervix ripening and the delicate relationships among these and other factors are discussed.

The data presented in the following Background paragraphs was collected from few leading reviews published in Clinical obstetrics and gynecology. Roy M. Pitkin and James R. Scott Eds. Volume 38, Number 2, 1995; and Volume 34, Number 4, 1991; and from a review article entitled “Nitric oxide, the endothelium, pregnancy and pre-eclampsia” published in the British Journal of Obstetrics and Gynaecology. January 1996. Vol. 103. pp. 4-15; all three are incorporated by reference as if fully set forth herein.

Premature rapture of the fetal membranes: Spontaneous rupture of the fetal membranes is often a normal component of the course of labor and delivery. This usually occurs after the onset of labor. It is considered premature rapture of membranes (PROM) if it occurs before the onset of labor. Fetal membrane rupture that occurs before the onset of labor at a gestational age of less than 37 weeks is preterm PROM (PPROM).

Incidence of PROM: The incidence of PROM ranges from 2-18%. Other, more. recent, reports show an incidence of 14-17%. These reported differences probably are influenced by population differences in contributory maternal and fetal risk factors. Approximately 60-80% of cases of PROM occur in term patients.

Significance of PROM: Because most patients with PROM deliver within 48 hours of membrane rupture, the significance of PROM depends on the gestational age of the fetus at its occurrence. With expectant management, approximately 9 of 10 term patients will progress spontaneously into labor with a latency period of no more than 48 hours. The latency period for PPROM is significantly longer, and the length varies inversely with gestational age. By 48 hours after PPROM, approximately 80% of patients whose fetuses are of gestational age 33-36 weeks and 66% of those with fetuses aged 20-32 weeks will undergo spontaneous labor.

Although most cases of PROM occur at term, its impact arises predominantly from the 20-40% of cases of PROM that occur before 37 weeks' gestational age. The significance of PROM is highlighted by the fact that 10% of the perinatal deaths among 53,000 pregnancies in the Collaborative Perinatal Project were found to be secondary to PROM.

The material impact of PROM is not as severe as its perinatal effects. Like the perinatal risks, maternal risks also may be affected indirectly and inversely by gestational age at the time of PROM. As the latency period extends to over 48 hours (as is more likely to occur with PPROM), the risk of antepartum and/or puerperal febrile morbidity increases. Because cesarean delivery is also a major risk factor for puerperal infection, management strategies to minimize the chance of abdominal delivery may be important in the presence of PROM. Expectant management of term PROM may decrease the risk of cesarean delivery and intraamniotic infection without increasing the rate of neonatal complications.

Mechanisms of PROM: Many risks factors have been identified for PROM. However, the final unifying mechanism for all cases must be the weakness in the chorioamnion membrane (relative or absolute, local or generalized) that allows rupture. To understand this weakness better, it is important to be aware of the factors that provide the normal integrity of the fetal membranes.

By the third trimester, the amnion consists of a single layer of epithelial cells. The chorion, and between these layers, there is a connective tissue zone, containing collagenous bundles, reticular fibrils, and fibroblasts.

With advancing gestational age, the fetal membranes undergo many stresses. Using direct measurement of fetal membrane surface area and indirect measurement of uterine surface area, investigators showed that the surface area of the uterine cavity is approximately twice that of the relaxed fetal membranes, implying a significant amount of membrane stretching in utero. Perhaps in response to this stretching, morphologic studies of term fetal membranes show a flattened and stretched appearance of amniotic epithelial cells at the sac position most distant from the placenta.

Morphologic changes in membranes from term patients with PROM are even more extreme than in those without PROM. The membranes are thinner near the rupture site, and the connective tissue layer contains a decreased number of poorly organized collagen fibrils.

Biochemical techniques also show a decline in the collagen content of prematurely ruptured amnion. In addition to the normal decline in the amnion's collagen with advancing gestational age, a further decline occurs in prematurely ruptured amnion Specifically, PPROM is associated with a reduction in the amniotic membrane content of type III collagen. Additionally, enhanced collagenolytic activity has been found in prematurely ruptured amniotic membranes.

Trypsin, a proteolytic enzyme present in amniotic fluid, preferentially degrades type III collagen. Amniotic fluid also contains alpha-1-antitrypsin as its primary antitryptic factor. After identifying the reduction of type Ill collagen in prematurely ruptured fetal membranes, one study found the amniotic fluid concentration of trypsin to be increased and that of alpha-1-antitrypsin to be reduced in women with PROM compared with the amniotic fluid from patients whose fetal membranes were not ruptured prematurely. Others repeated this study with a larger number of patients, better control of gestational age, and a more standardized method of amniotic fluid collection (amniocentesis rather than amniotomy). They found no relationship between the alpha-1 antitrypsin level and the occurrence of PROM.

Other potential sources of proteolytic insult to the chorioamnion membrane include proteases of seminal fluid, bacterial proteases secreted by cervicovaginal flora, and maternal proteases released in response to chorioamnionitis. The leukocytes that infiltrate the fetal membranes as part of the inflammatory response to chorioamnionitis may release elastase; this, like trypsin, selectively degrades type III collagen.

Evidence for deciduitis, chorioamnionitis, or intraamnionitic infection as causative of at least some cases of PROM includes the identification of clinically unapparent infection in approximately one quarter of women with PPROM who undergo diagnostic amniocentesis, and the increased risk of PROM in patients with group B betastreptoccocal cervical colonization, or cervicovaginitis caused by Bacteroides species or Trichomonas vaginalis.

Bacteria can weaken the fetal membranes, perhaps by protocolysis secondary to activation of the peroxidase-hydrogen peroxide-halide system in the fetal membranes and placental macrophages. Bacteroides Fragilis peptostreptococci and Fusobacterium, bacteria commonly isolated from amniotic fluid in the presence of preterm labor, and other common vaginal bacteria including Lactobacilli and Staphylococcus epidermidis have significant phospholipase A2 activity. Intrauterine bacteria also may lead indirectly to prostaglandin synthesis by activating macrophages. Intrauterine prostaglandin synthesis then may cause an increase in uterine contraction activity that also may weaken the fetal membranes.

The chorioamnion membrane behaves as a viscoelastic material, with characteristics of an elastic solid and a viscous liquid. Thus it undergoes deformation under stress, but it has imperfect memory. When the stress is removed, only the elastic deformation is recovered; this is less than the total deformation. Sequential deformations under stress, which might occur with physiologic or pathologic (preterm labor) uterine contractions, make the fetal membranes less tolerant of such stress and therefore more susceptible to rupture. As might be expected, term fetal membranes have different mechanical properties than those of preterm gestations. Preterm membranes that have undergone premature rupture tolerate more stress before rupture than do normal term membranes. Thus a mechanism other than overall membrane weakness must be postulated as an explanation fro PPROM. As an example of a local defect predisposing to rupture, samples of term membranes that underwent spontaneous premature rupture are thinner near the rupture site than membranes not subject to premature rupture.

These mechanistic principles may be combined to suggest that subclinical or overt intrauterine infection may, lead to uterine contractions, weakening the fetal membranes and perhaps augmenting a prior focal weakness, and thus causing at least some cases of PROM.

Cervix functioning: A pivotal question in the physiology of gestation and parturition is how one relatively small portion of the mammalian uterus, the cervix, functions at the anatomic and the molecular levels to retain the conceptus during gestation and to open sufficiently during parturition to allow the fetus to be delivered.

A remarkable dichotomy exists. On the other hand, the cervix, usually only three to four cm long, must remain firm and closed throughout pregnancy to ensure that the fetus is retained until fully developed and, therefore, able to survive outside the uterus.

On the other hand, this firm, unyielding portion of the uterus, consisting primarily of extracellular matrix, must dilate to a diameter of sufficient size, or about ten cm, to allow the fetus to be expelled from the uterus. The fact that the cervix is mainly connective tissue, predominately collagen type I and III, was first reported in 1947. Since then, scientific evidence has subsequently accumulated showing many aspects of the biochemical relationships of the phenomenal cervical function during pregnancy and birth.

During the past decade, the knowledge of cervical function has increased geometrically. Despite this, certain aspects of cervical physiology are unknown, especially the molecular biology of the trigger or triggers responsible for initiating the complex biochemical and biomechanical changes and rearrangement called “ripening”. There is also an increased effort to understand dilation as distinct from ripening.

Anatomy of the uterus: The uterus consists of three basic parts: the corpus or body and its endometrium, the isthmus and the uterine cervix. The uterus, a primarily muscular organ, is located in the pelvic cavity of nonpregnant women and of women during the first trimester of pregnancy. In later stages of gestation, it becomes an abdominal organ. It is situated between the bladder on its anterior surface and the rectum on its posterior surface. The whole of the nonpregnant uterus appears as a flattened pear. The caudal inferior portion of the uterus protrudes into the vagina and is approximately three cm long, two to three cm wide, and cylindrical in shape. It is called the cervix. The entire uterus is composed mainly of smooth muscle with extracellular matrix between the cells. The cervix, however, is only 10-15% smooth muscle on the average, with the remaining and larger percentage of tissue being connective tissue. The average size of the nulliparous uterus of an adult woman is 6-8 cm long: the average weight is 50-70 g. In multifarious women, the uterus measures 9-10 cm overall and weights approximately 80 g. The body of the uterus in young prepubescent girls is only half as long as the cervix, whereas in multifarious adult women the cervix and uterus are about equal in length, in multifarious women, however, the cervix is just a little more than one third the length of the whole organ. However, considerable individual variations exist in uterine size and shape among women.

The cervix is a cylindrical region of the uterus located at the caudal and inferior pole of the organ. It protrudes and opens into the vagina. It is bounded at its cephalic end by the internal os. The internal os is located at the peritoneal reflection of the bladder. The part of the cervix that protrudes into the vagina is called the portiovaginalis. The external os is connected to the internal os by a slender passage called the canal. Cervical mucosa is lined with tall columnar epithelium and contains many large, highly branched glands. These glands are lined by columnar epithelium, which ends abruptly at the level of the external os, giving way to the stratified squamous epithelium that covers the portiovaginalis and extends into the vagina proper. With age, the stratified squamous epithelium of the vagina may intrude past the external os into the lower portion of the cervix.

The underlying stroma of the cervix is predominately extracellular connective tissue matrix, namely type I and III collagen and a small amount of type IV collagen seen in the basement membranes. Water, glycosaminoglycans, and proteoglycans are important constituents of the uterine cervical matrix as well especially dermatan sulfate. Fibronectin, a different protein than fetal fibronectin, is present in the stroma also. Elastin, the functional protein of elastic fibers, is found in the cervix in physiologic amounts. The elastic fibers are 2-4 μm wide and run between the bundles of collagen fibers. They are located in a band that is 20-30 μm thick and parallel to a plane from the external os to the internal os under the epithelium of the port vaginalis and under the epithelium of the endocervical canal at the external os. These elastic fibers are very thin compared with elastic fibers of other tissue. Of importance is the fact that the ratio of elastin to collagen is highest at the area of the internal os. The greatest amount of smooth muscle is just below the internal os, and this smooth muscle tapers off toward the external os.

The uterine cervix serves as the channel through which the fetus must pass during normal birth. In a nonpregnant woman, the cervical canal is collapsed, fusiform, and firm, with the consistency of nasal cartilage. During pregnancy, its consistency becomes like the lips of the oral cavity. At approximately the 38th week of pregnancy, especially in multiparous women, the diameter of the endocervical canal at the level of the external os is often 1-2 cm. During labor, the cervix dilates to 10 cm in diameter to allow for the passage of the fetus. Cervical tissue, has considerable elasticity during labor and birth. Thus, during gestation and parturition, the uterine cervix undergoes, profound morphologic changes. Changes, or ripening, occur in gestation before labor begins.

Physiology: These profound changes are the result of a complex biochemical process involving many pathways and a rearrangement and realignment of the collagen. To a clinician, these processes result in softening or ripening, and their timely occurrence is essential for healthy parturition. Ripening that occurs too early in gestation often leads to preterm birth. Conversely, a, firm cervix, after full term, may contribute to dysfunctional labor. Cervical changes are also noticeable in early pregnancy, sometimes as early as one month after conception. At that time, vascularity and water retention increase, along with hypertrophy and hyperplasia of the cervical glands. These changes are responsible for the very earliest anatomic signs of pregnancy in women.

During the first stage of labor, mechanical stretching of the cervix occurs with the force of uterine contractions. Mechanical manipulation of the cervix in women causes a marked increase in blood levels of prostaglandin F2α, which in turn increases uterine contractions, a physiologic response called the Ferguson reflex.

The two differentiated parts of the uterus, the body and the cervix, and a portion of the uterus called the lower uterine segment, become even more distinctly separate in the first stage of labor. The contracting upper segment, or fundus, becomes thicker as labor progresses and the lower uterine segment and cervix become greatly expanded as time passes. The cervix and lower uterine segment are stretched and passively dilated by the presenting part of the fetus. The myometrium of the upper segment does not relax to its original length after a contraction but becomes fixed at a shorter length, with tension remaining the same as before the contraction. The consequence of this phenomenon has as advantage in the expulsion of the fetus, as each successive contraction commences where the previous contraction stopped, reducing the, volume of the uterine cavity and pushing the fetus slowly against the nonresistant ripened cervix and lower uterine segment.

The final change in the cervix is its effacement, or thinning of edges. This may occur early in the first stage of labor, or it may happen in the active phase. The timing of effacement varies among all women. The fetal membranes, amniotic sac, and fetal presenting part act as a wedge that allows dilatation and effacement to occur as the uterine contraction pushes them against the soft, nonresistant cervix.

Cervical Ripening:

Scientific evidence of the nature of cervical ripening has accumulated during the past several decades. Various types of experiments have been reported using both animal and human tissue. Although many vertebrate species have been used as models to study the anatomy and parturition, only the nonhumanoid primates have a reproductive tract similar to that of women. In understanding experiments using a rat model, it should be remembered that in this species the bicornate uterine horns are joined in the midline by the cervix. In adult rats, the uterus is composed of thick discrete bundles of collagen, smooth muscle, and elastin. The cervical tissue is mainly connective tissue matrix, consisting mostly of collagen glycosaminoglycans, and some elastin. The orientation of elastic fibers and smooth muscle in rat cervix is not clear at this time. The uterine collagen content increases between early and late estrus and decreases to the minimal amount at diestrus. Thus, it is controlled by hormones. Anatomically the reproductive tracts of other rodents are similar. Rabbits also have two uterine horns fused at the cervix. The endocrinology of pregnancy in rabbits is not completely typical of human pregnancy. However, guinea pigs are similar to humans in terms of the endocrinology of pregnancy-specifically, a shift of progesterone noted throughout gestation. Furthermore, the hormonal initiators of labor appear to be, similar to those of human. However, whether the extracellular matrix of the guinea pig cervix rearranges in a manner that is similar to that of human cervix is unclear.

The pregnant ewe is used extensively as a model for parturition, although the physiologic phenomenon of labor and its hormonal regulation is in many ways unlike that of human labor and delivery. In sheep, the dilation of the cervix is very quickly a few hours before spontaneous labor begins, or, alternatively, rapidly after induction of labor by infusion of corticotropin to the fetus.

Human cervical tissue is difficult to study for many reasons. Biopsy specimens samples area of the cervix, and the sample may not, and often is not, representative of the whole cervix. It is very difficult to obtain all of the cervix unless a total hysterectomy is done for appropriate reasons. Few studies reported in the literature have actually observed the complete cervix, and these either studied the nonpregnant organ or the rate cesarean hysterectomy specimen. The remaining studies report findings from small biopsy samples taken immediately after or sometimes before birth. Tissue obtained during cesarean section has been studied extensively in some cases. However, it's use is problematic in laboratories, because anatomic biopsy sites are not described in sufficient detail. The concern from the point of view of scientific accuracy is whether the cervix or the lower uterine segment was sampled. In addition, the cervix in pregnancy is very vascular. Biopsy specimens sample both stroma containing erythrocytes and leukocytes. Therefore, one must ask whether reported changes occur in the connective tissue itself or in blood vessels. These questions are not trivial from the scientific point of view. Ethical problems are also associated with sampling human cervix during pregnancy.

Human and mammalian cervical tissue has been studied in tissue culture and have provided much new knowledge, especially in developing our understanding of the biochemistry of matrix protein degradation, synthesis, and regulation. Normal cervical cells are difficult to grow in culture and usually require media enriched with fetal calf serum. Therefore the amount of growth factors is different than that is seen in living tissue. High collagenase production may be obtained in tissue culture, whereas in the living animal little or no collagenase is produced by the same tissue. When enzyme is extracted from tissue directly, low levels are observed. Furthermore, enzymes in tissue may be in an inactive form or bound to an inhibitor. Current scientific information appears to indicate differences in cervical ripening among species.

These technical concerns have created challenges for scientists studying cervical ripening and controversy over whether cervical ripening is predominately a biochemical rearrangement of the extracellular matrix, or whether the softening of the cervix is due to an inflammatory reaction where polymorphonuclear cells and macrophages release enzymes that degrade the collagen.

The role of smooth muscle cells in cervical ripening is beginning to be debated and studied.

Finally, the biochemical changes of clinical ripening need to be separated from the biochemical aspects of dilatation because these may be distinct processes.

Biomechanics of cervical function: Biomechanical studies of the cervix suggest a two-stage model of cervical modulation in gestation. During the first stage of labor, each contraction of uterine myometrium can dilate the cervix by as much as 30%. However, after realization of the uterus, and in the interval between contractions, most of the stretching is reversed, because the force generated by the uterus is reduced to zero. Therefore, the gradual opening of the human cervix from 2 to 10 cm is the result of a ratchet-like mechanism. Investigators think that the net gain of each contraction is approximately 2%. Progressive dilatation of the cervix is assisted by the viscoelastic behavior of this tissue during parturition. Mixed connective tissue containing elastin and collagen is viscoelastic. Viscoelasticity is a precise mechanical term and is characterized by stress relaxation, creep, and hysteresis. Stress relaxation occurs when tissue is suddenly strained at a constant level with time, the corresponding induced stresses (force) decrease with time. Creep is a phenomenon that occurs when tissue is suddenly stressed (a force applied). When stress is maintained, the tissue continues to deform. Hysteresis occurs when cyclic loading and unloading of a force occurs. The resulting loading stress-strain curve is different from the unloading curve.

Elastin is like rubber. As it stretches, entropy is lost with little decrease in energy. Therefore, when force is removed, the protein returns to its original length to maximize entropy. In the human uterine cervix, elastic fibers maintain the shape of the cervix and help to keep it closed. That elastin is important in maintaining pregnancy is shown by decreased elastin in women with an “incompetent cervix” a condition that can be thought of as a form of premature ripening and dilatation, although the mechanism is probably not identical to normal ripening. Elastic fibers contribute to the ratchet mechanism of dilatation. Elastin's ability to recoil helps to retain the fetus and also allows the cervix to regain its shape after birth.

Interstitial collagen fibers are tough and do not stretch easily. For collagen to exhibit tensile strength, two things are necessary. First, the collagen fiber needs to exceed a certain critical length; second, strong chemical bonds must exist between the collagen fibers and other matrix proteins. When these conditions exist, collagen is stiff and does not stretch.

Soft tissues are formed from a composite of extracellular matrix molecules and are arranges in various types of fiber networks, depending on the tissue. During pregnancy, normally the uterine cervix rearranges its collagen fibers. The nonpregnant cervix contains alignable collagen fibers that have a definite cable-like structure and form fibril bundles. These fibrils appear wavy when viewed with a light microscope. Proteoglycans form filaments that interact with collagen (and elastin) fibers and also act as a lubricant to the collagen fibers to slide by each other if stress is applied. Thus the collagen fibrils become aligned it the direction of the stress. Changes in the cervix during pregnancy cause a rearrangement of the collagen fibrils so that the tissue assumes the characteristics of a soft, easily dispensable tissue. In humans, apparently, this occurs in two ways. First, the concentration of hyaluronic acid increases, which attracts water molecules and contributes to softening of the tissue; second, hyaluronic acid increases in relation to dermatan sulfate. The decreased concentration of dermatan sulfate causes a decrease in the dermatan sulfate bridges between the collagen fibrils. X-ray diffraction shows a net loss in collagen fiber alignment and a decrease in fiber length to less than the critical length needed to assure great tensile strength. Polarized light studies of collagen fibers throughout gestation confirm this phenomenon. In other words, the extracellular matrix rearranges and contributes to the ripening process.

Connective tissue containing collagen and elastin has an incredible ability to rearrange its structure in response to a mechanical stress or force. The direction of the formation of collagen fibers and their location and their rate of synthesis are determined primarily by mechanical stress. A specific structural form of connective tissue can occur in response to a mechanical stress to suit a local physiologic function. In pregnancy, therefore, a predominately rigid, aligned, collagenous cervix retains the fetus in the uterus. Before parturition, this rigid structure must be modified. In late pregnancy, muscle fibers, fibroblasts, and collagenous and elastic tissues align in a definite direction, parallel to each other. This structural arrangement gives the collagen polarized strength. The mechanical pressure exerted on the cervix as the presenting part descends into the pelvis plays a role in both the realignment of the collagen and fiber bundles and in stretching the elastic fibers.

At full of term, before the onset of labor, the lower uterine segment measures about 4 cm wide, and its upper margin, the junction with the upper segment, is situated approximately at the level of the largest circumference of the presenting part of the fetus.

After the largest circumference of the presenting part of the fetus descents into the pelvis to the level of the ischial spines, it is said, to be engaged. After engagement, the pressure between the presenting part of the fetus and the uterine wall in the area of contact is as much as three to four times higher than the corresponding pressure in the amniotic cavity. This close contract between the fetus and the tissue of the cervix contributes to the 25-30 mm Hg that is usually needed to overcome the resistance of the mature, ripened cervix. A free gliding motion occurs between the presenting part of the fetus an the uterine wall, with a relatively low coefficient of friction. The fetal motion in the cervix is resisted by frictional forces, but factors reduce this friction. These factors are the vernix caseosa, amniotic fluid, blood and other secretions, and the motion of the fetus in the cervix that allows these lubricants to remain in the birth canal. Investigators have shown that pressure from the cervix and lower uterine segment prevents the descent of the fetal presenting part. This pressure corresponds to an amniotic pressure of 30-70 mm Hg with a fetal head of normal size. This vertex-to-cervix pressure at the largest circumference of the fetal head increases with the strength of the contractions and is higher in multiparous women than in nulliparous women. It is also higher after rupture of the chorion and amnion. The pressure of the presenting part to the cervix parallels the pressure generated by contractions. Thus, the dilatation of the normally ripened cervix in normal labor is passive. The biomechanics of cervical functions occur in two stages. First, the tissues reorient themselves in the direction of stress or deformation; second, the cervix dilates passively as the fetal presenting part is pushed into the cervix during the labor. The first stage of reorientation or alignment of the connective tissue components of the cervix is ripening.

Collagenases and other matrix metalloproteinases may contribute to this realignment, but they are not sufficient to initiate the process of ripening and may not be even necessary in the ripening of the uterine cervix in normal gestation. The matrix metalloproteinases may have functions in cervical physiology other then collagen breakdown of sufficient amount to cause ripening.

Biochemistry of the cervix during pregnancy: In adult tissue, synthesis and degradation of collagen and elastin occur slowly. However, in pregnancy the cervix becomes metabolically active. Water, an important component of the cervix in many species, increases substantially in pregnancy. This water interacts with the matrix proteins, is essential for the function of elastin, and greatly contributes to the clinical change in consistency in early pregnancy. In humans, however, there does not seen to be a change in water content immediately before or after delivery.

Elastin concentration does not appear to change in pregnancy. However, messenger RNA (mRNA) for tropoelastin (the precursor for elastin) is increased in pregnancy and again after birth.

Type I and type III collagen undergo marked change. As early as at 8-14 weeks' gestation, the spaces between collagen bundles become fibers, fibroblasts, and collagenous and elastic tissues of the cervix align in a definite direction, parallel to each other.

These structural alterations are accompanies by a decrease in collagen concentration. By full term, or immediately after birth, this concentration decreases by 30-50% compared with the nonpregnant cervix. However, this change in collagen concentration occurs because other components of the cervix are increasing. Water and noncollagen and nonelastin proteins are increasing, and at the same time the total amount of collagen is increasing, as shown by the fact that there is a marked increase in the level of type I collagen messenger RNA in pregnancy. The rate of collagen synthesis of other cervical proteins is even greater. The result is decreased collagen concentration. As noted before, collagen degradation by specific enzymes may not be an important factor in normal physiologic cervical ripening. However, recent radioactive labeling studies have shown that insoluble cervical collagen is degraded at the same time that collagen is synthesized. One explanation of this apparent contradiction is that the poorly formed collagen is being degraded by the cervical enzymes before it is truly incorporated into the tissue. An alternative explanation is that it is degraded somewhat and that this degradation of collagen and collagen rearrangement probably does play a greater role. Thus the degradation occurring is predominately newly synthesized collagen as part of the metabolic turnover process.

As gestation advances, collagen is easily extracted from cervical tissues, a phenomenon not observed in the nonpregnant state. Newly synthesized collagen is poorly cross-linked. These immature cross-liked contribute to ease in extraction of the newly formed collagen from tissues in the laboratory. Collagenase (now called matrix metalloproteinase-1) helps maintain a balance between collagen and degraded collagen and thus determines the amount of collagen laid down in tissue. This degradation of the poorly formed new collagen would regulate total collagen concentration. Thus the enzyme would not actually degrade the older collagen and well cross-linked, newly synthesized collagen, but rather would degrade poorly formed, scavenging collagen. Women with cervical incompetence have a low concentration of cross-linked collagen in tissues, which emphasizes the role of collagen in maintaining pregnancy.

An increasingly accepted theory of the change in cervical collagen is that a small proteoglycan, dermatan sulfate proteoglycan II (DSPG II or decorin), increases in the pregnant cervix. Decorin either coats the collagen fibrils and thus maintains the collagen at a smaller diameter, or it helps to separate the fibrils as it comes between the fibers of the collagen fibrils and opens up the bundles that contribute to their rearrangement. There are three dermatan sulfates, DSPG II or decorin, DSPG I or biglycan, and another called PGL. This increase might occur during ripening.

Hyaluronic acid concentration may decrease in pregnancy, remain unchanged, and increase. Species differences may exist. Alternatively, these differences can be explained by variations in the site of the biopsies obtained for analysis. In addition, more recently developed biochemical techniques may contribute to theses discrepancies. Newer evidence suggests that the decreased dermatan sulfate concentration occurs at the onset of labor and may be associated with progressive dilatation, whereas the concentration of hyaluronic acid increase nearly 12 times at 2-3 cm dilatation. Hyaluronic acid weakly interacts with collagen and fibronectin, and the increase of this glycosaminoglycan can help loosen the collagenous network of the cervix. Hyaluronic acid is an endogenous inducer of interleukin-1 synthesis in human monocytes and rabbit macrophages. This is an interesting finding given the role of cytokines in matrix protein metabolism.

Fetal fibronectin, a protein distinct from the fibronectin found in the extracellular matrix of the cervix, is synthesized by choriodecidual cells. Its presence in cervicovaginal secretions in the second and third trimesters identifies women at risk for preterm delivery. In some women at risk for preterm labor, fibronectin is released from the choriodecidual cells into cervical secretions. This phenomenon may reflect a separation of the chorion from the decidual layer of the uterus and the release of the fetal fibronectin into the secretions of the vagina and cervix. Recently it was shown to be a marker at full term for both spontaneous and prostaglandin E₂-induced cervical ripening. This fetal fibronectin is not involved in the biology of cervical ripening itself, but rather is found in the cervical secretions as a result of cervical softening. The presence of fetal fibronectin does suggest, however, that molecular events at the choridecidual internal os interface occur early in the course of cervical ripening. Studies show that fetal fibroncetin can predict preterm delivery as early as 4 weeks before its initiation. Ultrasound studies have shown a funneling of the internal os as a predictor of preterm labor. This is of interest because of the higher elastin/collagen ratio at the internal os than at other parts of the cervix, suggesting that pressure exerted by the presenting fetal part could contribute to a slight dilatation, which would allow the release of fetal fibronectin.

Role of hormones in cervical ripening: Prostaglandins regulate the components of the extracellular matrix in several ways. Prostaglandin F_{2α} increases one of the constituents of glycosaminoglycans and total glycosaminoglycan activity. In the case of cervical ripening, hyaluronic acid may induce the production of interleukin-1, a cytokine. Prostaglandin E₂ seems to dilate cervical small blood vessels, and it apparently produces a chemotactic response in leukocytes. Prostaglandin E₂ is used extensively to soften an unripe cervix. Clinicians understand the efficacy of such treatment to facilitate delivery well. Prostaglandin E-induced cervical ripening is associated with a time-limited enzymatic collagen degradation, increased synthesis, of noncollagenous proteins, and a substantial increase in hyaluronic acid concentration.

Estrogen increases collagen concentration in skin. Unopposed estrogen in oophorectomized rats increases the uterine concentration of the elastin cross-link, desmosine, and the collagen cross-link, hydroxyproline. It can initiate process of programmed cell death, called apoptosis, in the cervix. Cervical softening has been reported to occur during pregnancy when an increase in plasma 17β-estradiol was observed. The cervical ripening process in women is associated with apoptosis.

Dehydroepiandrosterone sulfate concentration is higher in plasma if women with clinically ripening cervices. Given as an injection to pregnant women at 38-42 weeks, dehydroepiandrosterone sulfate causes a marked improvement in cervical softening. The effect of this hormone may be due to its metabolite, estradiol-17-β, although a direct effect of dehydroepiandrosterone sulfate on the production of procollagenase and prostromelysin by fibroblasts from pregnant rabbit uterine cervix was observed.

Progesterone changes cervical softening. Well-conducted studies in guinea pigs, using the progesterone antagonists onapristone, lilopristone, and nifepristone, and the progesterone agonist promegestone, show that the biochemical changes of cervical softening, including a decrease in collagen and glycosaminoglycan concentration, are mediated by an apparent progesterone receptor. In tissue and cell culture, progesterone receptor and estrogen decrease the amount of procollagenase (matrix metalloproteinase-3) and their mRNAs, whereas the amount of tissue inhibitors of matrix proteases (TIMP-1 and TIMP-2) is increased by these hormones.

Therefore both estrogen and progesterone regulate the biochemical modulation of the uterine cervix as the pretranslational level of the specific protein.

Relaxin, an ovarian hormone released during gestation, softens the cervix of rodents and decreases delivery time in rats. In humans, there are two relaxin genes, H₁ and H₂. H₁ is expressed by the human ovary, while H₁ and H₂ are: produced by both decidua and trophoblasts. In rats, preliminary evidence suggests that relaxin treatment correlates with apoptosis. Relaxin can soften the human cervix, but the exact molecular mechanism of relaxin's role is unknown.

Effect of cytokines on cervical ripening: The theory of the role of inflammatory cells in the rearrangement of the extracellular matrix proteins of the cervix was proposed more than 10 years ago. Since then, many studies have focused on proving this theory. Dissolution of the connective tissue matrix has been reported around polymorphonuclear leukocytes in the uterine cervix after delivery. In both light and electron microscopy, a “halo” was seen surrounding the infiltrating neutrophils. The assumption was that this was sue to a degree of collagen degradation, although it could also be due to rearranged collagen.

Studies have reported the presence of activated leukocytes and eosinophils in cervical biopsy specimens at full term. Cytokines, especially interleukin-1 play a role in cervical ripening. Cervical fibroblasts in culture release interleukin-8, a known chemotactic factor for neutrophils that softens the cervix in guinea pigs and rabbits. The precise role of inflammatory cells in the physiology of the cervix during gestation and parturition is not completely understood. Perhaps cytokines such as interleukin-8 draw neutrophils into cervical tissues near the end of the first and second stages of labor. Inflammatory cells have not been observed in the ripening process in some animal models, and there is no well-designed study in humans that shows unequivocally that infiltration of inflammatory cells contributes to cervical ripening in normal pregnancy. Inflammation does, however, play a definite role in causing preterm labor.

Role of matrix metalloproteinases and their endogenous inhibitors: Reproductive hormones control the production of collagenase and other proteases that can degrade extracellular matrix proteins in vitro. They appear to affect the synthesis of matrix proteins directly at the pretranslational level. They also seem to have a direct effect on the synthesis degradative enzymes. Thus there appears to be the fine balance, as previously discussed, between the synthesis of procollagen message and that of the procollagenase message. Complicating the picture is the role of matrix protease inhibitors, especially TIMP-1 and TIMP-2 (tissue inhibitors of matrix protease). TIMP-3 has been identified in other tissues. TIMP is increased by estrogen and progesterone.

Recently, much has been learned about the proteolytic enzymes that degrade extracellular matrix proteins. These enzymes comprise a family of 12 matrix metalloproteases. Nine of these MMPs have been identified in humans. Each enzyme requires zinc as cofactor. MMP-1 is tissue and macrophage collagenase and is secreted in an inactive form. When activated it cleaves collagen types I, II, and III into three-quarter to one-quarter fragments. It degrades type III collagen 16 times faster than does MMP-8 or neutrophil collagenase.

Both connective tissue cells and inflammatory cells secrete matrix metalloprotease in the active form. Enzymes are activated in a stepwise of cascade fashion. Complete dependents on the presence of procollagenase activators. The procollagenase activator is MMP-3, or stromelysin, which is activated by many enzymes, including an elastase-like enzyme. Elastase has been found in cervical tissue, and it is regulated by estrogen and increases in the cervix at full term in normal pregnancy. Its function might be to activate MMP-3, and thus a cascade phenomenon occurs in the process of degradation of the cervical collagen. The important point is that the degradative pathways of the extracellular matrix proteins are complex and finely regulated.

In abnormal situations, however, such as preterm labor, these enzymes play a greater role than in normal ripening processes. Both cervical inflammation and chorioamnionitis will initiate the release of matrix metalloproteases.

Role of smooth muscle cells: During pregnancy, smooth muscle cells of the cervix become enlarged and prominent. Increased amounts of smooth muscle has been reported in human tissue obtained from women with clinically incompetent cervices. Changes in cervical smooth muscle may play a role in cervical tissue rearrangement and orientation. Collagen bundles are aligned in close approximation to the smooth muscle bundles. Recently investigators showed that apoptosis, or programmed cell death, is seen and associated with the changes of cervical softening, at least in rat cervical tissues. Apoptosis is a phenomenon characterized by cell shrinkage, compaction of chromatin into nuclei. It is induced by physiologic stimuli such as estrogens and other steroid hormones, as well as cytokines. Apoptosis occurs in isolated cells so that many metabolically active cells are intermixed with dying cells. This type of cell death occurs dysynchronously. Cervical apoptotic cells show oligonucleosomal length fragmented DNA is visualized in situ when digoxigenin-labeled DNA detected. The role of apoptosis in cervical ripening is not understood completely. The rat is the only species that has been reasonably well studied. There is some suggestion that apoptosis occurs in humans. However, the phenomenon has been poorly observed until now. It may be postulated that the increased disorganization of collagen bundles combined with the decrease in the myofibrils of smooth muscle cells, causes cervical softening. An appealing aspect of apoptosis is that it is genetically timed. Cervical ripening occurs in a timely, species-specific manner. Therefore, because normal parturition is time specific for each species, cervical cells death may be genetically programmed as a physiologic event. The relationship at a molecular level between the death of cervical smooth muscle cells and the rearrangement of the collagen bundles of the cervix is speculative: Apoptosis is characterized by intact lysosomes of the dying cells, as opposed to necrosis where lysosomes can leak degradative enzymes into surrounding tissue. Despite this, in electron micrographs, cervix collagen bundles in close proximity to dying smooth muscle cells appear to be disorganized, whereas other collagen bundles located farther away from dying cells are intact.

Local changes in sex hormones, including estrogen and progestin, at the cellular level, might activate the gene that regulates degradation of the DNA in the smooth muscle cells leading to cell death. Perhaps also these dying cells are stimulated to synthesize cross-linked collagen type I and III collagen. Evidence shows that injured lung fibroblasts and macrophages express increased levels of transforming growth factor beta, a cytokine. The dying smooth muscle cells of the cervix might produce cytokines that then stimulate cervical fibroblasts and/or other cells to produce matrix-metalloproteinases, which are seen in active labor. Many future experiments will be necessary, however, to elucidate the exact role of programmed cell death in the biochemical change of cervical ripening. The demonstration that apoptosis exists in the cervix in late gestation provides exciting new avenues of search.

Thus, the uterine cervix is a unique organ composed predominately of the extracellular matrix proteins, collagen, elastin, and glycosaminolycans. During pregnancy and labor, this organ is metabolically active, which is different than in adult tissue. The metabolism is under reproductive hormonal control and is more complex than previously appreciated.

Smooth muscle cells, which comprise 10-15% cervical tissue, undergo programmed cell death and play a role in cervical softening. Apoptosis is genetically timed event and could explain the species specific length of gestation. Further research in the next several years will reveal more completely the exciting process of cervical ripening will can then be more accurately diagnosed and treated. For example, if apoptosis is shown to play an important role in the process of cervical ripening, it could be inhibited. Conversely, it could be induced in the unripe cervix. If we would look for it, we would find that it is probably occurring today in the clinical use of cervical ripening agents.

The most important contributor to cervical softening, however, is a rearrangement and realignment of the collagen, elastin, and smooth muscle cells, which occurs due to mechanical forces and to a rearrangement of the collagen that occurs as the content of glycosaminoglycans varies in the cervix with time. One form of dermatan sulfate, decorin, may help to separate the collagen fibrils and then open them up. This rearrangement also involves fiber shortening below the critical length for tensile strength, allowing for extendibility that the cervix undergoes during dilatation. Finally, the cervix undergoes change in two phases-softening, which involves collagen realignment, and dilatation. The proteolytic enzymes in the cervix degrade cross-linked, newly synthesized collagen, and they help activate other enzymes in a cascade. However, the predominate anatomic and physiologic change in ripening is the rearrangement of collagen.

Oxytocin: Induction of labor implies the initiation of uterine activity to effect labor and delivery. Modern obstetrics offers three principal methods to induce labor amniotomy, prostaglandin compounds, particularly E₂ and F_{2-α,} and oxytocin. The following discussion addresses the last method.

Oxytocin is one of the most frequently used compounds in modern obstetrical treatment, but it is used cautiously, since the potential for maternal and fetal compromise exists. Oxytocin is the only uterotonic agent with U.S. Food and Drug Administration approval to induce labor with a viable fetus. A thorough understanding of the pharmacokinetics and clinical effects of oxytocin will allow the most efficient use of oxytocin and prevent untoward complications.

Oxytocin and vasopressin (antidiuretic hormone) are the two hyothalamic neurohormones released by the posterior lobe of the pituitary gland. Oxytocin is synthesized in the paraventricular and supraotopic nuclei by the formation of large precursor molecules that are cleaved and stored in the neurohypophysis. Oxytocin is short neuropeptide consisting of nine amino acid residues with a disulfide bridge between two cysteine residues in positions 1 and 6, giving the molecule a ring structure. Integrity of the disulfide bridge is essential of biologic activity. The oxytocin peptide structure differs from vasopressin only in the presence of isoleucine rather than phenylalanine in position 8. The similarity in structures accounts for the small antidiuretic and vasoactive activity produced by oxytocin when used in large doses.

Oxytocin circulates in an unbound form and is cleared from the maternal circulation by the kidney and the liver. Oxytocinase, a circulating cystyl-aminopeptidase, is produced by the human placenta and rapidly degrades oxytocin in vitro. The metabolic clearance of oxytocin is increased during gestation when Oxytocinase activity is high. Oxytocinase cleaves at the cystein-tyrosine bond between positions 1 and 2, eliminating biologic activity by destroying the ring structure. The plasma half-life of oxytocin is relatively brief, from 5 to 17 minutes.

Oxytocin is released, in a pulsatile fashion, in response to various stimuli that increase the firing rate of neurons in the paraventicular nucleus of the hypothalamus. Breast stimulation leads to oxytocin-induced contraction of mammary myoepithelial cells (the milk-ejection reflex). Sensory stimuli from the lower genital tract and cervical stretching (Ferguson reflex) also affect oxytocin release and uterine contractions. Oxytocin appears to stimulate production and release of arachidonic acid and PGF2-α by decidua that has been appropriately sensitized to oxytocin. This potentiates oxytocin-induced uterine contractions.

In addition to milk ejection and uterine stimulation, other systemic oxytocin effects occur. Another action is direct vascular, smooth muscle relaxation. Vasodilatation can be substantial, although transient, in response to a large infusion dose. Bolus intravenous administration of oxytocin may result in hypertension, reduced coronary perfusion and cardiac arrest. These effects are most pronounced in the patient under anesthesia. Due to the antidiuretic activity of oxytocin when used in large doses, in the presence of excessive intravenous fluid administration water intoxication has been described. Relative to vasopressin, oxytocin has 1% of the antidiuretic and pressor activity. When used in physiologic doses, side effects are minimal.

Some investigators found the circulating level of oxytocin in pregnancy to increase slightly from, nonpregnant levels, whereas others have not. Radioimmunoassay studies indicate that circulating oxytocin is released in a pulsatile fashion and can be measured on the maternal peripheral blood in “spurts”. There appears to be stabilization between late pregnancy until the first stage of labor. During labor, oxytocin levels during the first stage of labor are consistent with those produced by an intravenous oxytocin infusion rate of 2 to 4 mU/min. Dawood and colleagues, by measuring oxytocin concentrations in the umbilical artery and vein, determined that during spontaneous labor oxytocin is produced in the fetal compartment and contributes to the increased maternal levels measured.

There is increasing sensitivity of the myometrium throughout gestation. Myometrial responsiveness to oxytocin begins at about 20 weeks and increases steadily until a marked rate of increase at 30 weeks, reaching a maximum in spontaneous labor at full term. Coincident with the increased sensitivity of the uterus to oxytocin is an increase in the concentration of oxytocin receptors in the myometrium and decidua in late pregnancy. Receptor levels are maximal after the onset of labor, whether at full term or preterm, and were found by Fuchs and colleagues to be even higher that levels seen just before the onset of labor. The clinical implication is that the uterus is insensitive to the uterotonic effects of oxytocin until substantial oxytocin receptor concentrations are induced. Oxytocin receptors are sparse in the human cervix, thus uterine sensitivity to oxytocin does not imply cervical ripening.

The physiologic processes involved in the initiation and progression of labor are complex and incompletely understood. It is clear, however, that the role of oxytocin is pivotal. The human myometrium is particularly sensitive to the hormonal influences of estrogen and progestins, which appear to level. The function of oxytocin is probably both direct and facilitating. That is, oxytocin stimulates the receptor-rich uterus to contract and causes the release of decidual prostaglandins, further enhancing uterine contractility.

Oxytocin induces membrane rapture: The development of safe, and effective regiments for oxytocin administration during the second half of this century has provided long-desired control of the onset of labor. Indeed, the ability to effect safe and timely delivery underlies the rationale for maternal and fetal monitoring during the third trimester of pregnancy. In the absence of the ability to induce labor, the only alternative to expectant management is cesarean birth. Although the relative safety of cesarean now allows for facile, if less than optimal, management, quite recently the risks involved were much greater. Then, as today, a dilute solution of synthetic: oxytocin, was the obstetrician's most powerful and most dangerous tool. Furthermore, cesarean birth, although considered fairly routine in many developed areas, remains a substantial strain on the limited medical resources available in many areas of the world.

The optional dosage, interval of increase in concentration, and even pulsatility of administration continue to be debated. Despite this continued fine-tuning, however, familiarity, safety, and reliability has maintained oxytocin as the agent of choice to induce labor.

Deficiency of oxytocin infusion in unfavorable cervix: The major deficiency of oxytocin infusion to induce labor is a high failure rate in women with an unfavorable cervix. After the acceptance of oxytocin during the 1950s, experience with induction fostered some important conclusions. As noted by Turnbull in Britain and Bishop in the United States, oxytocin infusion, with or without concomitant membrane rupture, results in a high rate of failed induction if the cervix is not “ripe”. The importance of cervical readiness can be appreciated by the effort expended by many investigators to establish methods for accurate assessment. As seen in the contribution of Fuentes and Wiliams, these efforts have continued and now include measurements based on sonography. Unfortunately, use of cervical scoring systems, such as those based on the studies of Bishop, often underscores the frustration to be expected if induction is attempted in women in whom the gradual process of effacement and dilation has not begun.

The lack of efficiency of oxytocin in women with low cervical scores has led to investigation of other agents and methods of labor induction, both chemical and mechanical. Amniotomy, the oldest method to induce labor, is most effective in women with very favorable cervical scores and poses a substantial risk for infection because of the unpredictable interval between membrane rupture and active-phase labor. As reviewed by Busowski and Parsons amniotomy alone or in conjunction with oxytocin in women with an unfavorable cervix leads to a 24 hour interval to rupture and cesarean birth in more than one half the patients so managed.

Prostaglandins: Labor is induced by causing uterine myometiral contraction before their spontaneous onset, which stimulated the cervix to efface and dilate to allow subsequent passage and birth of the fetus. Sometimes labor induction is a difficult obstetrical problem, such as in the post-term nulliparous women with an unfavorable cervix.

The condition of the cervix, as originally described by Bishop, is the most important factor for the successful induction of labor. Bishop's score is the single most reliable predicator of vaginal delivery in attempts to induce labor. Based on this concept, cervical preparation (ripening) before labor is induced has gained tremendous attention in the obstetric community.

An important differentiation between cervical ripening and induction of labor must be made, because regular strong uterine contractions are not only unimportant in the ripening process but sometimes could even be considered an unwarranted side effect of a ripening method. Cervical ripening refers to a prelabor phase when the cervix changes characteristics (such as consistency, position, effacement, and dilatation), whereas induction refers primarily to attempts to produce regular uterine contractions along with cervical changes to begin the active phase of labor. In clinical practice, however, the two terms often have many overlapping features, and the difference becomes relatively unimportant compared with the ultimate outcome of successful vaginal delivery without fetal or maternal compromise.

Agents used to induce labor produce a phase of cervical change during the induction. This phase is much shorter than when the same agents are only used for ripening. Therefor, the distinction between cervical ripening and labor induction is sometimes artificial and includes the obstetrician's intention, drug dosing, and intensiveness.

Most obstetricians in the United States are familiar with oxytocin to induce labor. However, oxytocin, used in the traditional manner, is not always sufficient to induce labor and other drugs and mechanical methods have been developed for use in conjunction with oxytocin. Unfortunately, no single methods or protocol has been proved uniquely effective. Most inducting agents are compared to various oxytocin protocols or placebo.

Prostaglandins E₂ (PGE₂) and F_{2-α} (PGF_{2-α}) are powerful oxytocin agents. They were introduced in the late 1960s, although their properties were known before. Prostaglandin agents have been used in intravenous, oral, vaginal, intracervical, and extravular routes to induce labor. Intravenous and oral administration of the various prostaglandin preparations was introduced in the 1970s, and the transvaginal route was made popular in the early 1980s. Much scientific investigation has been done using various forms of prostaglandin. Gordon-Wright and colleagues analyzed, in a prospective, randomized study, the effectiveness of prostaglandin E₂ tablets administered intravaginally using increasing doses from 1 to 5 mg to induce labor. A placebo control group was also included. With progressively increasing doses, more patients were induced successfully (achieving active labor), ranging from 29.6% in the 1-mg group to 62.9% in the 5-mg group in primigravidae. In multigravidae, the range was 39.1% to 62.2%, respectively. The investigators observed a dose-related response. In the same study, a 1-mg PGE₂ suppository was more effective than placebo (which induced labor in only 7.4% of those treated). In almost all of the dose categories, a change in Bishop score of more than 3 points was observed more frequently in primigravidous compared with multifarious women. Labor induction was slightly more successful in the multifarious groups. Neither hypersonic contractions nor other maternal side effects were observed.

In other prospective, randomized study, McKenzie and associates used 5-mg vaginal suppositories for primigravidae and 2.5-mg suppositories for multigravidae and compared them to women in a placebo group. The PGE₂ group showed greater success in induction (63% of the primigravidea and 81% of the multigravidae established labor and delivered their infants without oxytocin augmentation), less need for additional oxytocin (37.5% compared with 100% in primigravidae, and 19.1% compared with 100% in multigravidae), and shorter treatment-to-delivery interval (7.6 to 11.6 hours) compared with controls. Neither hypertonous nor gastrointestinal side effects were observed in any of the patients studied. In addition, postpartum hemorrhage occurred less frequently in the PGE₂ group compared with the control group.

In 1984, in prospective randomized trial, Campbell showed that the success rate for induced labor was 55% when 3-mg PGE₂ intravaginal pessaries were used in a mixed group of multigravidae and primigravidae, which confirms previous reports. A significant change in the Bishop score was observed in both primiparas and multiparas. Campbell detected no side effects, including hypersonic contractions.

Meta-analysis of all suitable trials comparing vaginal PGE₂ with placebo to induce labor shows that induction failed less frequently in the treatment group than in the placebo group (typical odds ratio, 0.14 [0.09-0.22]). Side effects did not differ from controls.

Because the efficacy of vaginal Prostaglandins was shown to be superior to placebo, studies subsequently compared this treatment with traditional oxytocin induction.

Macer and coworkers randomized 85 patients to receive either 3-mg PGE₂ suppositories or intravenous oxytocin. After the single suppository alone, labor was achieved in 98% of patients in a mean time of 1.5 hours. However, additional oxytocin augmentation was needed in 71% of nulliparous 14% of multifarious women. Duration of oxytocin was shorter in the PGE₂ gropup compared with the group receiving only oxytocin. No difference on the vaginal delivery, operative delivery, or cesarean section rates were found between the groups. Side effects were similar and minimal.

Andearsson and colleagues, in a study from Denmark, randomized pregnant women needing induction to receive 3-mg PGE₂ group, higher delivery rates were achieved in the first 24 hours compared with those in women receiving oxytocin. Failed induction, cesarean section rates, and complications were similar.

Ekman and associates, in a Swedish trial comparing women who received either 3-mg PGE₂ vaginal suppositories or intravenous oxytocin, found that PGE₂ was more effective for promoting vaginal birth within 24 hours (17 of 19 women) than was oxytocin alone (8 of 19 women) in women with cervical Bishop score of 4-5.

Meta-analysis conducted with all appropriate trials comparing vaginal Prostaglandins with oxytocin to induce labor concluded that failed induction and no vaginal birth within 12 or 48 hours occurred less frequently with Prostaglandins than with oxytocin. Side effects were similar.

When compared with oxytocin alone, intracervical insertion of PGE₂ (dose, 0.4-1 mg) effectively induced labor, similar to intravaginal applications in women with low Bishop scores. In women with ripe cervices, on the other hand, no differences between the women who received PGE₂ and those who received only oxytocin only were found.

Nowadays, the most commonly used agents for nonmechanical induction of labor are prostaglandins of the F and E series. These agents have been administered in vaginal suppositories, appears to offer some advantages over oxytocin to induce labor, especially in women with unfavorable cervical scores.

Disadvantages, however, including systemic side effects and difficulties with control of administration have prevented prostaglandin E from gaining widespread popularity as the sole agent to induce labor.

Prostaglandins, however, have achieved considerable attention when used to help begin induction in women with unfavorable cervices. This popularity is so great and confidence in the safety of prostaglandin E gel so high that there is now considerable experience with this agent in women with previous cesarean sections who have induced labor.

Nitric oxide: Nitric oxide is an inorganic free radical gas which, over the last decade or so, has been shown to possess more potential biological functions than any known molecule. This wide-ranging biological activity reflects the importance of nitric oxide as a modulator of cellular activity. It has important vasoactive functions related to its ability to inhibit platelet aggregation and relax perivascular smooth muscle. Nitric oxide also functions as a neurotransmitter, and has been implicated in the pathogenesis of a spectrum of diseases, including septic shock and chronic hypertension. It has an unpaired electron in its outer orbital and, in pure form, in both solid or liquid phases, achieves chemical stability by forming dimers. The unpaired electron makes the molecule highly reactive and it readily combines with oxygen to produce nitrogen dioxide, a powerful oxidizing agent.

Nitric oxide generation by nitric oxide, synthase (NOS) isoenzymes which produce nitric oxide from the essential amino acid L-arginine, have a significant role in the regulation of the vascular endothelium in pregnancy. Abnormalities in nitric oxide synthesis could contribute to the development of pregnancy-induced hypertensive disorders and therefore modulation of nitric oxide availability has potential therapeutic roles.

Discovery and characterization of nitric oxide: It was demonstrated that the vascular endothelium was not merely the inert lining of blood vessels, but that it was able to influence adjacent smooth muscle in the vessel wall. Removal of the endothelial monolayer from the vessel prevented the production of a relaxing factor, thereby producing contraction. This substance was named endothelial-derived relaxing factor (EDRF) with a half-life of seconds. Its effect on vessel relaxation was blocked in the presence of oxyhaemoglobin and enhanced in the presence of the enzyme superoxide dismutase. Endogenous vasoactive substances including bradykinin, histamine, serotonin; adenine, nucleotides and shear stress, have all been shown to result in the production of EDRF.

In 1987 it was suggested that EDRF was NO because the two compounds had very similar biological properties. Shortly after it was shown that EDRF release from cultured cells required the essential amino acid L-arginine. Subsequently, it was shown that L-arginine analogues inhibited nitric oxide release from the vascular endothelium.

Nitric oxide has a short half-life and is able to diffuse easily across cell membranes due to its solubility in both water and lipid, enabling it to act as a cell-to-cell messenger. The target for nitric oxide synthesized in a generator cell is soluble guanylate cyclase, an enzyme which catalyses the formation of guanidine cyclic monophosphate (cGMP). Nitric oxide interacts with the heme moiety of guanylate cyclase, activating the enzyme and thereby increasing the intracellular concentration of cGMP. This intracellular second messenger in turn activates protein kinases, which in smooth muscle cells leads to dephosphorylation of the myosin light chains and relaxation.

Nitric oxide syntheses: Nitric oxide is synthesized from L-arginine by a family of enzymes, most of which are cytosolic, known as the nitric oxide synthases (NOS). These proteins have features in common with cytochrome P450 reductase and contain both oxidative and reductive domains. The production of nitric oxide, which also results in the formation of the amino acid L-citrulline, requires molecular oxygen and at least four cofactors, namely protoporphyrin, flavin mononucleotide, flavinadenine dinucleotide and tetrahydrobiopterin. NOS is readily inhibited by L-arginine analogues such as N-methyl-L-arginine (L-NMMA); N-nitro-L-arginine; and N-nitro-L-arginine methyl ester (l-NAME). NOS is also inhibited by flavoprotein binders, and calmodulin binders. Enzyme activity is oxygen-dependent, and it has been shown that a reduction in oxygen saturation will reduce nitric oxide synthesis.

Three isoforms of nitric oxide synthase have now been identified. Of these the endothelial and neuronal isoforms are constitutive, i.e., they are always present. They are activated by a flux of calcium into the cells. The constitutive endothelial isoform (eNOS) is found in both large and small vessel endothelium, in platelets and is probably released continuously from both arterial and arteriolar vascular endothelium in healthy tissues. An infusion of an L-arginine analogue (which inhibits enzyme activity) into the brachial artery results in a substantial fall in resting forearm blood flow. This suggests that the basal tone of arteries and arterioles is dependent upon continuous synthesis of nitric oxide. Systemic infusion of an NOS inhibitor increases arterial blood pressure in healthy subjects but does not affect venous pressure since veins do not have a basal nitric oxide release. Pulmonary arteries also synthesize nitric oxide continuously, and presumably it is a part of the mechanism which ensures matched ventilation and perfusion. Nitric oxide synthesized by the vascular endothelium causes not only vasodilation but, like prostacyclin, also decreases the affinity of the endothelium for platelets (i.e., it produces thromboresistance), thereby contributing to homeostasis. Platelets, once activated, release serotonin and bradykinin which stimulate eNOS activity in healthy endothelium, preventing excessive platelet aggregation and adhesion to endothelium.

The constitutive neuronal isoform is found in both central and peripheral neurons. A non-adrenergic non-cholinergic nitrergic nervous system, with nitric oxide as a neurotransmitter, has now been proposed and nerves staining for NOS have been found in the cardiovascular system, bronchial tree, urinary tract and the gastrointestinal tract. Nitretic nerves may well play an important role in the dilation of certain blood vessels, and also the relaxation of gastrointestinal sphincters, including the sphincter of Oddi. NOS can be demonstrated in nerves throughout the brain, being found most frequently in the cerebellum, and superior and inferior colliculi.

The main site of the inducible isoform is found in the macrophage and is produced in response to infection, bacterial endotoxin, exotoxin, or cytokines such as IL2 and TNF. It is relatively independent of calcium for its activity. Macrophage-derived nitric oxide is cytotoxic to a number of pathogens including fungi, protozoa and mycobacterium tuberculosis. The genes for these isoforms have been mapped to chromosome 7 (endothelial), chromosome 12 (neuronal) and chromosome 17 (macrophage).

The evidence for altered nitric oxide production in pregnancy: Initially it was showed that acetylcholine-induced relaxation of isolated guinea pig uterine arteries was not only dependent on the presence of an intact endothelium, but also on the release of a nitroso-like compound, which relaxed smooth muscle. Having identified this as nitric oxide, the effects of pregnancy and sex steroids, on calcium-dependent and calcium-independent NOS activity was examined. It was demonstrated that in late pregnancy in the guinea pig there was a four-fold increase in calcium-dependent NOS activity in the uterine artery and a doubling of activity in the heart, skeletal muscle, oesophagus and cerebellum was inhibited by tamoxifen, an estrogen receptor antagonist. Estrogen therapy in the non-pregnant guinea pig also resulted in an increase in calcium-dependent activity. Northern blot analysis showed that there was an increase in mRNA for calcium-dependent NOS in both pregnant and: estrogen-treated animals, suggesting that the rise in NOS activity resulted from estrogen-mediated enzyme induction.

Then it was demonstrated that plasma levels and urinary excretion of cGMP increases in pregnancy in the rat. Subsequently it was shown that there is increased urinary excretion of the stable nitric oxide oxidation product, nitrate, and the presence of nitrosohemoglobin in blood—a metabolite not seen in pseudopregnancy or in a non-pregnant group was demonstrated. Furthermore, it was confirmed that nitric oxide synthesis is increased in pregnancy, since the infusion of a potent NOS inhibitor prevented the usual rise in urinary excretion of nitrate and cyclic GMP.

In addition, it was demonstrated that the L-agrinine-NO-cGMP pathway system is functional in the rat uterus and works to inhibit contractility during gestation. L-arginine and nitric oxide donors (compounds capable of releasing nitric oxide in vivo) were used to produce myometrial relaxation. Subsequently it was demonstrated to be similar in human myometrium. By examining myometrium obtained at different gestational ages, age-dependent changes in the effects of L-arginine and nitric oxide donors on relaxation were found, with increasing concentrations of either L-arginine or cGMP being required to induce myometrial relaxation as pregnancy progressed. These findings suggested that the L-arginine-NO-cGMP pathway regulates uterine contractility throughout gestation. There is also a reduction in myometrial and decidual NOS activity in late pregnancy which may contribute to the accompanying increase in uterine activity.

Reduced total NOS activity in rat myometrium immediately prior to parturition and 80% decrease in decidual calcium-independent NOS activity on the last day of pregnancy in the rabbit were also reported. Recently it was shown that NOS activities were significantly reduced in human myometrium obtained late in gestation compared to non-pregnant controls, and that an 80% reduction in amniotic fluid nitrite concentrations characterizes late pregnancy, although the source of the nitric oxide was not determined. It was further shown that there is an L-arginine-NO cGMP pathway in the human uterus, and that there is a decrease in uterine relaxation responsiveness to nitric oxide at term, which may play a role in the initiation of labor.

NOS was localized to the syncytiotrophoblast cell layer in human, placenta. Significant calcium-dependent and calcium-independent NOS activity in human placental villi and in the basal plate. Was reported, but minimal NOS activity was shown to be present in the placental bed. It was found that the placental vascular tree synthesized a predominantly calcium-dependent isoform of the enzyme, while the calcium-independent activity represented only 6% of the total. An immmunohistochemistry approach was recently employed to show that endothelial NOS at term was localized in the endothelium of the umbilical artery and vein; staining patterns were also, strong in the placental syncytiotrophoblast but they were more variable in the chorionic vessels and were absent in the endothelium of the small feto-placental vessels and cytotrophoblast cells. Placental endothelium NOS has now been purified and characterized. RT-PCR was used to demonstrate that the mRNA encoding for inducible NOS is resent in the placenta.

Role of the vascular endothelium in pregnancy: The vascular endothelium in a healthy adult female weighs approximately 1.5 kg, and there is sufficient pulmonary vascular endothelium to completely cover six football pitches. The endothelial surface is constantly exposed to hormonal factors, inflammatory mediators, and changes in shear stress. Shear stress, occurring secondary to changes in blood flow, may well be the most important stimulus for nitric oxide release. It was demonstrated over a decade ago that the removal of the endothelial monolayer markedly reduced flow-induced vasodilation. The functions of the vascular endothelium are, therefore, not only to monitor both hemodynamic and hormonal signals, but also to modulate the release of vasoactive substances which act to regulate thromboresistance and tone in the vessel wall. The endothelium acts to inhibit blood coagulation by synthesizing and secreting thrombomodulin and heparan sulphate onto its luminal surface, and modulates fibrinolysis by synthesizing plasminogen activators and inhibitors. Endothelial-derived prostacyclin is well known to contribute to platelet inhibition and to vasodilation. In contrast, endothelial-derived constricting factor, or endothelin, will induce vasoconstriction.

Normotensive human pregnancy is associated with pronounced cardiovascular changes, including an increase in heart rate, cardiac output and blood volume, and a decrease in arterial pressure and responsiveness to angiotensin II. These cardiovascular changes have, until recently, been attributed to the increased production of endothelial-derived vasodilator prostaglandins acting to regulate blood pressure during pregnancy. Estrogens also appear to be involved in pregnancy-associated vascular refractoriness and nitric oxide has been shown to mediate estrogen-induced vasodilatation. An animal model was used to demonstrate that nitric oxide is probably an important regulator of maternal blood pressure. It was shown that chronic infusion of N-nitro-L-arginine, an NOS inhibitor, increased mean arterial blood pressure and reversed the pregnancy-induced refractoriness to angiotensin and vasopressin in rats. It was further shown that prolonged blockade of nitric oxide synthesis produced a pre-eclampsia-like syndrome with the development of sustained hypertension, a reduced intravascular compartment, thrombocytopaenia, proteinuria and fetal growth retardation and demise.

Normotensive pregnancy is associated with a hugely increased blood flow in the uteroplacental circulation in parallel with the developing fetoplacental circulation. On the material side, the uteroplacental bed is progressively transformed from a high-pressure system to a low-pressure, high-flow system in order to meet the requirements of both the placenta and the developing fetus. On the fetal side, the normally low fetoplacental perfusion pressure occurs as a result of the release of vasoactive substances, and the appropriate anatomical development of the distal branches of the fetal villous tree. Under normal conditions, therefore, there is a continuous forward flow velocity in the umbilical artery, which suggests a low impedance to flow in the placental circulation.

Nitric oxide is also important in regulating fetoplacental blood flow. It was shown that infusing an NOS inhibitor (nitro-L-arginine) into the umbilical artery of chronically catheterized sheep resulted in an increase in fetoplacental vascular resistance and a resultant reduction in umbilical artery blood flow. In the human, it was demonstrated that the isolated perfused term placental lobule, preconstricted with the thromboxane mimetic, U46619, could subsequently be vasodilated by nitric oxide donors. In a subsequent study, it was shown that L-NAME (a potent NOS blocker) increased fetoplacental perfusion pressure in vitro. Further support for the role of nitric oxide in the fetoplacental circulation was provided by the use of small vessel myography to show that L-NAME significantly reduces flow-induced dilatation in placental arteries. Collectively, these findings suggest that trophoblast-derived nitric oxide contributed to be control of placental vascular tone.

Pre-eclampsia: The development of pre-eclampsia begins with a loss of vascular refractoriness to vasoactive agents, followed by vasoconstriction. A functional imbalance between vasodilator and vasoconstrictor eicosanoid products appears to be of major importance in causing this loss of vascular refractoriness. Patients who develop pre-eclampsia exhibit a smaller increase in prostacycline. (PGI₂) biosyntheses than normal and a reduction in the urinary excretion of PGI₂ metabolites precedes the development of clinical disease Thromboxane A₂ (TXA₂) biosynthesis is increase in pre-eclampsia, and the urinary excretion of TXB₂ metabolites correlates with the severity if the pre-eclampic disease process. The absence of the normal stimulation of the renin-angiotensin system, despite the significant hypovolaemia and the increased vascular sensitivity to angiotensin-II and norepinephrine, can be explained by a single mechanism: endothelial cell injury causes a deficiency in the production and/or activity of vasodilator prostaglandins, and in particular, that of PGI₂. The resulting increased TXA₂-to-PGI₂ ratio may be the cause of selective platelet destruction (sometimes accompanied by microangiopathic hemolysis), while reduced uteroplacental blood flow is the result of spiral artery thrombosis and placental infarction. The normal physiological adaptation of the spiral arteries does not occur in pre-eclampsia, or is limited only to the decidual portion of the spiral vessels, and many of the spiral arteries are occluded by forbinoid material and surrounded by foam cells.

Although the concept of a PGI₂-TXA₂ imbalance provides an explanation for many of the clinical feature of pre-eclampsia, this concept is now being challenged. It was observed that vasodilator prostaglandins do not mediate the changes in renal hemodynamics or the attenuation of the systemic and renal pressor responsiveness observed during normal pregnancy. More recently, indomethacin was used to block prostaglandin synthesis during human pregnancy and it was shown that it had no effect on vascular resistance. A correlation between the urinary excretion of PGI₂ metabolites and angiotensin I sensitivity was not found. Vasodilator prostaglandins may, however, provide part of a rescue mechanism when tissue perfusion has become endangered. It was found that the plasma concentrations of PGI₂ metabolites were higher in women who had a marked response to angiotensin-II. The plasma concentrations of PGI₂ metabolites were highest in the group remaining angiotensin-II sensitive after low-dose aspirin therapy. These findings suggest that vascular PGI₂-release occurs as a result of platelet aggregation and thrombin production to prevent further vascular damage.

The rat model has been used extensively to examine the role of nitric oxide in pregnancy. Studies in spontaneously hypertensive pregnant rats suggested that nitric oxide was the major antihypertensive factor rather than the vasodilator prostaglandins. In this animal model, the physiological decrease in blood pressure observed in normal pregnancy appeared to depend completely on endothelial nitric oxide release, while vascular prostacyclin synthesis was riot found, to be important. In other animal studies, inhibitors of cyclo-oxygenase failed to alter pressor responsiveness.

There is now substantial evidence for endothelial cell dysfunction in pre-eclampsia. It was shown that serum obtained from pre-elamptic women has a greater cytotoxic effect on cultured endothelial cells than serum from normotensive pregnant women (and also a greater mitogenic effect on fibroblasts cells). It was also suggested that pre-eclampsia involves endothelial cell dysfunction. Morphological evidence of endothelial injury is provided both by the characteristic kindly lesion of pre-eclampsia, known as glomerular endotheliosis, and by the ultrastructural changes in the placental bed and uterine boundary vessels. It was further suggested that there is an increase in the mitogenic effect of plasma samples from women in the first trimester of pregnancy who subsequently developed pre-eclampsia, and that cellular fibronectin levels also increase at the beginning of the second trimester. Material plasma levels of endothelin are elevated in pre-eclampsia, but this neither precedes development of the disease nor correlates with its severity. However, it does probably reflect extensive maternal endothelial damage.

Increased levels of factor VIII-related antigen (von Willebrand Factor), fibronectin, cellular fibronectin and thrombomodulin have all been reported in pre-eclampsia. These substances are markers of endothelial cell activation. An imbalance between tissue plasminogen established disturbance are also thought to be contributory factors.

The causes of the endothelial cell dysfunction seen in pre-eclampsia are, however, still unclear. It has been proposed that pre-eclampsia is a two-stage placental disease with the first stage being attributed to abnormalities in the normal processes which affect uteroplacental blood supply. The second stage is believed to encompass the effects of the resulting placental ischemia on the fetal and maternal circulation.

In the placenta, the maternal blood is in direct contact with the placental syncytiotrophoblast, a multi-nucleated true syncytium with an extensive microvillous brush border. In pre-eclampsia, the microvilli of the syncytioblast are abnormally shaped, and there are focal areas of necrosis. It was shown that trophoblast deportation is greatly increased in pre-eclampsia. Syncytiotrophoblast microvillious membranes were isolated and it was shown that these could inhibit endothelial cell growth in vitro, with the suggestion that sloughed-off microvilli may be responsible for the development of the maternal syndrome of pre-eclampsia due to the resulting endothelial cell damage.

An alternative explanation for the symptoms of pre-eclapsia is that there is an immune maladaptation mechanism resulting in endothelial cell damage and dysfunction. The decidua is mainly lymphoid tissue and it is possible that activated decidual neutrophils release substances causing endothelial damage. These agents include the contents of neutrophil granules, such as elastase and other toxic proteases, as well as cytokines and oxygen-free radicals which can all disturb the integrity of the endothelial cells, vascular basement membrane and subendothelial matrix. Leukotrienes, which are synthesized and released following neutrophil activation, can cause an increase in vascular permeability, induce vasoconstriction, and promote further neutrophil activation and adherence. Neutrophil activation, localized in part to the placental bed, has been demonstrated to occur in pre-eclampsia. It was shown that in pre-eclampsia neutrophil activation enhances the production of the free radical superoxide. It was further shown that VCAM-1 which is a soluble cell adhesion molecule and a marker of endothelial damage and neutrophil activation, is selectively elevated in serum in pre-eclampsia.

Platelet activation is a physiological feature of healthy pregnancy, and is exaggerated in pre-eclampsia. Excessive platelet activation could be responsible for the disseminated intravascular coagulation seen in the disease. In pre-eclampsia, the number of circulating platelets is reduced and they are larger in size, indicating increased platelet consumption. The platelet count has been shown to fall in the pre-clinical phase of pre-eclampsia. Platelet reactivity is inhibited by cGMP and in vivo studies have shown that nitric oxide will stimulate platelet guanylate cycles activity, and is therefore a potent inhibitor of platelet activation. An in vitro model was used to show that platelets obtained from pre-eclamptics are more susceptible to the inhibitory effects of nitric oxide donors. This may have occurred as a secondary response to impaired vascular nitric oxide generation. In pre-eclampsia, platelets are more prone to adhering to the endothelium and releasing alpha- and dense granule constituents: TXA₂ and serotonin are then generated, contributing to platelet aggregation and inducing the formation of fibrin to stabilize platelet thrombi which may eventually occlude maternal blood flow to a placental cotyledon, leading to placental infarction. The increased levels of circulating, platelet-derived serotonin induce further platelet aggregation, and may also amplify the vasoconstrictor action of certain neurohumoral mediators, in particular catecholamines and angiotensin-II, thereby causing direct contraction (via S₂-receptors) of vascular smooth muscle.

However, not all the evidence currently available points: conclusively in the same direction. While it was shown that plasma nitrite levels were significantly lower in patients with pre-eclampsia and that there was a negative correlation between serum nitrite levels and diastolic blood pressure in patients with pre-eclampsia, in contrast, no difference was found in the plasma concentration of nitrites in these groups, whereas elevated plasma nitrites was found in women with established pregnancy-induced hypertension.

Plasma concentrations of ADMA (asymmetric dimethyl L-arginine), an endogenous arginine analogue which probably functions as an NOS inhibitor, are raised in patients with chronic renal failure. It was recently shown that the plasma concentration of ADMA was increased in pre-eclamptic women compared with normotensive women, or women with pregnancy-induced hypertension.

Growth-retarded fetuses frequently demonstrate reduced, absent or even reversed uterine artery blood velocities during diastole. It was observed that placentae from pregnancies complicated by absent end diastolic flow velocities had reduced numbers of small arterioles within small stem villi. Studies in the fetal sheep confirmed that embolization of the fetoplacental circulation could also reduce umbilical blood flow, but the relationship was not linear since impedance was only gradually increased. There has to be a substantial increase in the vascular impedance before abnormal Doppler artery waveforms can be demonstrated in the human fetoplacental circulation. Hypoxia will also contribute to increased vasoconstriction, and it was shown in a human placental perfusion model that acute reduction of the oxygen tension in the maternal perfusate can cause vasoconstriction. Endothelial regulation of fetoplacental vascular tone may also be abnormal in pregnancies complicated by growth retardation.

Reduced EDRF activity was demonstrated in umbilical vessel perfusates in pre-eclampsia compared to normal controls. Recently, a marked decrease was found in umbilical L-arginine levels in fetuses from pre-eclamptic women with absent and/or reversed end-diastolic umbilical blood flow. According to these findings, low L-arginine levels may be of pathophysiological importance in these fetuses, resulting in decreased umbilical NO release. Reduced cGMP levels in the placental circulation in pregnancy-induced hypertensive disorders was also shown.

Lower NOS activities was shown in placental villi in pregnancies complicated by pre-eclampsia and growth retardation compared to villi from normal placentae, whereas, using stem villous arterioles from placenta from pregnancies exhibiting abnormal flow velocity waveforms, could not demonstrate impaired endothelial dependent relaxation. This may be due to the reduction or absence of eNOS in some of the smaller placental vessels even in normal placentae, and/or the persistence of eNOS in some vessels even in pre-eclampsia, such that measurements of single vessels may not be representative of overall placental function.

Therapeutic perspectives in the management of pre-eclampsia and of premature rapture of fetal membranes: A nitrovasodilator is a generalized term for a therapeutic agent which releases NO in vivo and thereby stimulates cGMP synthesis. These include nitrogen-containing compounds such as glycerol trinitrate (GTN), the inorganic nitrates, such as sodium nitrite, and nitrates and compounds such as sodium nitroprusside. Sodium nitroprusside decomposes spontaneously to release nitric oxide, while GTN is enzymatically metabolized into nitric oxide. Administration of nitric oxide donors reduces the size of myocardial infarctions in animals and has been used successfully in hypertensive crisis to reduce ventricular after-load. However, the exact mechanism for this effect is not known and nitrate tolerance may occur after long term treatment.

Sodium nitroprusside has been used in the management of severe hypertension in pre-eclamptic patients. Unlike hydralazine, it has a powerful but brief anti-hypertensive action, and is an extremely potent vasodilator. Wasserstrum reported that circulatory distress and paradoxical bradycardia can develop following its use in pre-eclamptic patients who have not been pre-treated with plasma volume expansion.

De Rosayro and colleagues investigated the effects of intravenous GTN administration to normotensive and hypertensive ewes GTN caused a reduction in uterine blood flow as a result of a decrease in blood pressure, apparently with no adverse fetal effects. Wheeler found that GTN reduced mean arterial blood pressure, but without altering uterine blood flow, and prevented the expected increase flow that should have resulted from giving noradrenaline. There have now been several studies using GTN to treat patients with established proteinuric hypertension. Cotton showed that GTN reduced mean arterial blood pressure by 25%, and capillary wedge pressure by 30%, without any significant change in heart rate, central arterial pressure or stroke volume in patients with severe pregnancy-induced hypertension. They also showed that plasma volume expanders had no effect on mean arterial pressure, but that the combination of blood volume expansion arid GTN resulted in a marked resistance alone.

Giles and colleagues have reported changes in the umbilical artery blood velocity waveforms (suggesting reduced resistance) following GTN administration. Similarly, Gruewald administered intravenous GTN to patients with severe pre-eclampsia and found changes in the umbilical artery. They also observed a significant reduction in blood pressure during the infusion, but did not find any alteration in the Doppler flow velocity waveforms in the uterine arteries. In contrast, Ramsey found that intravenous GTN, given in the first trimester, increased uterine artery diastolic blood velocity in normal early pregnancy, mimicking the physiological alteration of the uterine artery flow velocity waveform which is seen with advancing gestation. However, GTN administration caused only a non-significant increase in the uterine artery velocities in women with abnormal uterine artery Doppler measurements at 24 weeks, and did not alter the umbilical artery flow velocity waveform.

GTN patches have now been used in the management of preterm labor and were reported to be a “safe, well-tolerated and non-invasive method of suppressing preterm labor”, although only 13 cases were studied. Randomized trials will be needed in order to determine whether there is any genuine therapeutic benefit of nitric oxide donors in the prevention and management of preterm labor, and until the results of these are available, use of GTN should be confined to such trials.

S-nitroglutathione (GSNO) is a nitric oxide donor and a potent inhibitor of platelet activation at doses that do not lower blood pressure, and has been used in the treatment of HELLP syndrome. A GSNO infusion for 90 minutes resulted in a rapid improvement of the patient's hematology, liver biochemistry and renal function.

N-acetycysteine (NAC) is a glutathione (GSH) precursor and a sulphydryl group donor. Recent studies have shown that NAC enhances nitric oxide production from GTN, and potentiates the hypotensive action of acetycholine through a nitric oxide-dependent mechanism. NAC will enhance the vasoactive and anti-platelet activity of nitric oxide donors by the formation of S-nitrosothiol, which protects nitric oxide from being metabolized by free-radical scavengers. NAC and the combination of NAC and nitric oxide donors appear to be an interesting option for further clinical research on the prevention and management of pre-eclampsia.

Plasma L-arginine is present in large amounts in the plasma and its availability is (theoretically) unlikely ever to be the rate-limiting step in the formation of nitric oxide by the endothelium. L-arginine supplementation in women with normal endothelial and renal function therefore seems unlikely to be useful, as a result of which there are few published studies of such a strategy. However, it has been speculated that L-arginine availability may be a factor in diseases where there is increased nitric oxide degradation in dysfunctional endothelium. Fetal arginine levels are lower in pregnancies complicated by IUGR. Raij have shown in pregnant rats that L-arginine supplementations will prevent glomerular thrombosis. Rossitch have similarly demonstrated that isolated vessels from atherosclerotic animals exhibit enhanced of L-arginine. Orally administered L-arginine has recently been shown to increase exhaled nitric oxide in normal women, and the authors have suggested increasing the nitric oxide in diseases in which where is defective nitric oxide production. Studies of the effects of L-arginine supplementation in pregnant women with pre-eclampsia might therefore be worthwhile.

As is evident from the above discussion a number of factors take active part in the processes of fetal membranes rapture, cervix ripening and labor. Low progesterone/estrogen ratio, elevated levels of prostaglandins, collagenase and other matrix metalloproteinases, cytokines and oxytocin, and lowered levels of nitric oxide, which functions as muscle relaxant, all act to induce membranes rapture, cervix ripening and labor.

Although commutative evidence exist for the functionality of these factors during the processes membranes rapture, cervix ripening and labor, the prior art fails to teach a multidrug approach for inhibiting these processes, so as to influence more than a single factor influencing them.

There is thus a widely recognized need for, and it would be highly advantageous to have, a method and pharmaceutical composition that act on a number of different factors to prevent premature rapture of the fetal membranes, cervical ripening and preterm labor.

### SUMMARY OF THE INVENTION

According to the present invention there are provided a method and a pharmaceutical composition for inhibiting premature rapture of the fetal membranes, ripening of the uterine cervix and preterm labor in female mammals.

According to further features in preferred embodiments of the invention described below, the method comprising the step of administering compounds for reversing at least two biochemical conditions being associated with ripening of the fetal membranes.

According to further features in preferred embodiments of the invention described below, the pharmaceutical composition comprising compounds for reversing at least two biochemical conditions being associated with ripening of the fetal membranes.

According to still further features in the described preferred embodiments the biochemical conditions are selected from the group consisting of high level of collagenase activity, high level of cytokines, low ratio of progesterone effect versus estrogen effect, low level of nitric oxide, high level of prostaglandins effect and high level of oxytocin effect.

According to still further features in the described preferred embodiments (a) reversing the high level of collagenase activity is effected by a collagenase inhibitor; (b) reversing the high level of cytokines is effected by an anticytokine antibody or a cytokine carrier; (c) reversing the low ratio of progesterone effect versus estrogen effect is effected by a first substance selected from the group consisting of progesterone, a progesterone receptor agonist and an estrogen receptor antagonist; (d) reversing the low level of nitric oxide is effected by a nitrovasodilator; (e) reversing the high level of prostaglandins effect is effected by a prostaglandin receptor antagonist; and (f) reversing the high level of oxytocin effect is effected by a second substance selected from the group consisting of oxyticinase and an oxytocin receptor antagonist.

According to still further features in the described preferred embodiments the collagenase inhibitor is selected from the group consisting of caffeic acid, hydroxyquinoline, hydroxyquinoline derivative, phosphonepeptide, benzyloxy carbonyl-specified peptide sequence, a peptide sequence, anticollagenase antibodies, tri-peptide hydroxamic acid derivative, CaNa₂EDTA, alpha-2-macroglobulin, alpha-1-antitripsin, a metalloprotease inhibitor, a cysteine proteinase inhibitor, N-acetyl-cysteine, N-acetyl homocysteine, N,N′-diacetylcystine, L-arginine, guanido substituted arginines or homoarginines, L-arginine N^{G} alkyl derivative, glycerol trinitrate and tissue inhibitor of matrix protease.

According to still further features in the described preferred embodiments the anticytokine antibody is selected from the group consisting of anti interleukin 1, anti interleukin 2, anti interleukin 6, anti interleukin 8 and anti tumour necrosis factor.

According to still further features in the described preferred embodiments the anticytokine antibody is selected from the group consisting of a polyclonal anticytokine antibody and a monoclonal anticytokine antibody.

According to still further features in the described preferred embodiments said cytokine carrier is alpha-2-macroglobulin.

According to still further features in the described preferred embodiments the nitrovasodilator is selected from the group consisting of glycerol trinitrate, L-arginine, guanido substituted arginines or homoarginines, L-arginine N^{G} alkyl derivative, N-acetyl-cysteine, N-acetyl homocysteine, N,N′-diacetylcysteine, an inorganic nitrate, a nitrate, sodium nitroprusside and alpha 1 adrenergic antagonist.

According to still further features in the described preferred embodiments the prostaglandin receptor antagonist is indomethacin.

According to still further features in the described preferred embodiments a conventional substance used for inhibiting premature rapture of the fetal membranes, ripening of the cervix and preterm labor is further employed.

According to still further features in the described preferred embodiments the conventional substance is selected from the group consisting of treatment with MgSO₄, beta mimetic, Ca blocker, an oxytocin receptor antagonist, Atosiban and antibiotics.

According to still further features in the described preferred embodiments the beta mimetic is selected from the group consisting of salbutamol, ritodrin and indomethacin.

According to still further features in the described preferred embodiments the compounds are in a form selected from group consisting of creme, ointment, gel, liquid, spray, powder, pill, capsule and patch.

According to still further features in the described preferred embodiments said administration is effected via a route selected from the group consisting of subcutaneously, intravenously, intramuscularly, orally, intracervically, intramniotically, extramniotically and intravaginally.

According to still further features in the described preferred embodiments the pharmaceutical composition further comprising a substance selected from the group consisting of thickeners, carriers, buffers, diluents, surface active agents and preservatives.

The present invention successfully addresses the shortcomings of the presently known configurations by providing a method and a pharmaceutical composition that act synergistically on a number of different factors to prevent premature rapture of the fetal membranes, ripening of the uterine cervix and preterm labor in female mammals.

### DESCRIPTION OF THE PREFERRED EMBODIMENTS

The present invention is of a method and a pharmaceutical composition for inhibiting premature rapture of the fetal membranes, ripening of the uterine cervix and preterm labor in female mammals. The present invention can be used to lower the risk of premature membranes rapture, cervical ripening and preterm labor by, for example, prophylactic administration of the inventive pharmaceutical composition to a pregnant female mammal, including woman, or administration of the inventive pharmaceutical composition following a preterm labor and membrane rapture test, as described, for example, in U.S. Pat. Nos. 5,096,830; and 5,641,636; both are incorporated by reference as if fully set forth herein.

The principles and operation of the method and composition according to the present invention may be better understood with reference to the descriptions below.

Collagen is a naturally occurring protein found in humans and animals. collagen is one of the most abundant proteins in mammals and 50%-70% of collagen is found in the cervix.

Just before and during preterm labor and during interm labor collagenase enzyme increases dramatically in the tissues and in the circulation, the blood levels being 60-70 ng/ml in active labor or during preterm labor. During labor and until the end of delivery the amount of collagen in the cervix, which provides the required rigidity during gestation, is usually significantly reduced. The collagen in the cervix and in the lower segment of the cervix is degraded as a result of increase in the amount of collagenase.

Therefore, according to one aspect of the present invention a collagenase inhibitor is employed to inhibit the reported collagenase activity and to prevent premature. membranes rapture, cervical ripening and preterm labor. Please note that collagenase inhibitors are used for prevention of collagenase induced diseases, see, for example, U.S. Pat. No. 4,276,284.

Cytokines, such as, but not limited to, interleukines and tumour necrosis factor, are naturally occurring proteins found in humans and animals, which are typically secreted from cells of the immune system and act both as chemotaxis compounds and as activators of other immune system cells to inflict various immune responses.

During preterm labor and during interm labor the level of cytokines increases in the tissues and in the circulation and function to attract and activate cells of the immune system resulting in dissolution of the connective tissue matrix in the cervix, which leads to ripening.

Therefore, according to another aspect of the present invention a cytokines effect inhibitor is employed to inhibit the reported activity and to prevent premature membranes rapture, cervical ripening and preterm labor.

Progesterone and estrogen are the main hormones governing conception, gestation and labor. Low progesterone versus estrogen ratio is considered the main trigger of labor. Thus, during preterm labor and during interm labor the level of progesterone increases in the tissues and in the circulation and functions to initiate and sustain processes which eventually lead to membranes rapture, cervical ripening and labor.

Therefore, according to yet another aspect of the present invention a progesterone effect inhibitor is employed to inhibit the reported activity and, to prevent premature membranes rapture, cervical ripening and preterm labor.

Nitric oxide (NO) is an endogenous molecule involved in many bodily processes. NO acts as a muscle relaxant which may inhibit the muscle contractions associated with membranes rapture, cervical ripening and labor. The association of NO with diseases related to vasoconstriction is disclosed in U.S. Pat. Nos. 5,132,407; 5,266,594, 5,273,875; 5,281,627 and 5,286,739, all of which are incorporated by reference as if fully set forth herein.

Therefore, according to yet another aspect of the present invention a nitrovasodilator is employed to increase the level of NO and thereby prevent premature membranes rapture, cervical ripening and preterm labor.

Prostaglandins are endogenous hormones involved in the process of membranes rapture, cervical ripening and labor. Just before and during membranes rapture, cervical ripening and labor the blood and cervical levels of prostaglandins increases dramatically.

Therefore, according to still another aspect of the present invention a prostaglandin effect inhibitor is employed to reduce the prostaglandins effect and thereby prevent premature membranes rapture, cervical ripening and preterm labor.

Oxytocin is an endogenous hormone involved in the process of membranes rapture, cervical ripening and labor. Just before and during membranes rapture, cervical ripening and labor the blood and cervical levels of Oxytocin increases dramatically.

Therefore, according to another aspect of the present invention an oxytocin effect inhibitor is employed to reduce the oxytocin effect and thereby prevent membranes rapture, cervical ripening and preterm labor.

The association of progesterone, estrogen, prostaglandins, collagen and collagenases, cytokines, oxytocin, nitric oxide and other factors: with membranes rapture, cervical ripening and labor, and the complex relationships thereamongst are further detailed in the Background section above.

Thus, according to one embodiment of the present invention provided is a method of preventing premature membranes rapture, cervical ripening and preterm labor in a pregnant female mammal including humans. The method includes the step of administering the mammal with compounds for reversing at least two biochemical conditions associated with membranes rapture, cervical ripening and labor.

This treatment, will prevent and/or stop premature membranes rapture, cervical ripening and preterm labor. Since at risk women are hard to prognose, the invention can beneficially be used, for example, in preterm (e.g. thirty seven week) gestation by prophylactic administration of the compounds into the cervix of pregnant females and/or; in the serum or following a preterm labor and membrane rapture test.

According to another embodiment of the present invention provided is a pharmaceutical composition for preventing premature membranes rapture, cervical ripening and preterm labor in a pregnant female mammal. The composition includes compounds for reversing at least two biochemical conditions associated with membranes rapture, cervical ripening and labor.

The term “reversing” as used herein in the specification and in the claims section below refers to reducing or increasing (depending on the context) the biochemical condition.

According to a preferred embodiment of the invention the biochemical conditions are high level of collagenase activity, high level of cytokines, low ratio of progesterone effect versus estrogen effect, low level of nitric oxide, high level of prostaglandins effect and high level of oxytocin effect.

The term “effect” is used herein and in the claims below to imply that the substance's effect is mediated via a high affinity receptor to the substance.

The terms “high” and “low” are used herein and in the claims below to indicate the level of the condition (e.g., concentration, magnitude, etc.) during membranes rapture, cervical ripening and labor, as opposed to its level during gestation before these events take place. Thus, a condition which is defined high during labor is low before labor and vice versa, whereas the change in the level of the condition is associated with membranes rapture, cervical ripening and labor.

According to the present invention, reversing the high level of collagenase activity is preferably effected by administration of a collagenase inhibitor. The collagenase inhibitor is preferably caffeic acid, hydroxyquinoline, hydroxyquinoline derivative, phosphonepeptide, benzyloxy carbonyl-specified peptide sequence, a peptide sequence as for example described in U.S. Pat. No. 4,371,466, which is incorporated by reference as if fully set forth herein, anticollagenase antibodies, tri-peptide hydroxamic acid derivative, CaNa₂EDTA, alpha-2-macroglobulin, alpha-1-antitripsin, a metalloprotease inhibitor, a cysteine proteinase inhibitor, N-acetyl-cysteine, N-acetyl homocysteine, N,N′-diacetylcysteine (see U.S. Pat. No. 4,724,239, which is incorporated by reference as if fully set forth herein), L-arginine, guanido substituted arginines or homoarginines (see U.S. Pat. No. 5,281,627, which is incorporated by reference as if fully set forth herein), L-arginine N^{G} alkyl derivative (as, for example, described in U.S. Pat. Nos. 4,499,068; and 5,059,712) which is incorporated by reference as, if fully set forth herein), glycerol trinitrate (nitro-glycerine), tissue inhibitor of matrix protease or any combination thereof. Additional collagenase inhibitors are available from Boehringer Mannheim and are listed in the 1996 biochemicals catalog thereof on pages 460-465, which are incorporated by reference as if fully set forth herein. The collagenase inhibitors administered may augment the naturally produced inhibitors in the cervix and in the systemic circulation.

Should alpha-1-antitrypsin be the collagenase inhibitor of choice, it can be administered intravenously, intramuscularly, via an aerosol or extraamniotically, as taught by a press release of Bayer Corporation dated Sep. 2, 1998, published in YAHOO FINANCE.

The particular pharmaceutical carrier used will vary depending on the form of the pharmaceutical composition and the intended method of administration as further detailed hereinbelow. The pharmaceutical composition may be administered by injection of alpha-2-macroglobulin to the systemic circulation, in such amount that will block locally the collagenase that would otherwise further digest and ripen the cervix e.g., 4-6 grams of CaNa₂EDTA in the cervix and the upper vagina or in combination with alpha-2-macroglobulins in systemic circulation. Administration should be such to reach levels of CaNa₂EDTA of about 0.1 M in the systemic circulation or ideally intravenously.

According to the present invention, reversing the high level of cytokines is preferably effected by administration of an anticytokine antibody, a cytokine carrier, such as alpha-2-macroglobulin, or a cytokine receptor. The anticytokine antibody is, for example, anti interleukin 1, anti interleukin 2, anti interleukin 6, anti interleukin 8 and anti tumour necrosis factor (TNF) which is naturally present amniotic fluid. The anticytokine antibody may be a polyclonal anticytokine antibody and/or a monoclonal anticytokine antibody. The anti tumour necrosis factor (TNF) antibody can be a conjugate of an antibody an a TNF receptor. Alternatively, it can be a TNF receptor, as recently developed by Immunex Corporation and described in a press release dated Aug. 4, 1998, published in YAHOO FINANCE.

According to the present invention, reversing the low ratio of progesterone effect versus estrogen effect is preferably effected by administering a substance such as progesterone, a progesterone receptor agonist and an estrogen receptor antagonist or any combination thereof.

According to the present invention, reversing the low level of nitric oxide is preferably effected by administration of a nitrovasodilator. The nitrovasodilator can be, for example, glycerol trinitrate, L-arginine, guanido substituted arginines or homoarginines, L-arginine N^{G} alkyl derivative, N-acetyl-cysteine, N-acetyl homocysteine, N,N′-diacetylcysteine, an inorganic nitrate, a nitrate, sodium nitroprusside and alpha 1 adrenergic antagonist (see U.S. Pat. Nos. 4,282217; 4,734438; 5,028,627; and 5,059,712, teaching alpha 1 adrenergic agonist for use to reduce NO levels).

According to the present invention, reversing the high level of prostaglandins effect is preferably effected by administration of a prostaglandin receptor antagonist, such as, but not limited to, indomethacin.

According to the present invention, reversing the high level of oxytocin effect is preferably effected by administration of a substance, such as, but not limited to, oxytocinase and an oxytocin receptor antagonist.

Collagenase enzyme inhibitors for use in the present invention are readily obtainable and are used as medications to treat other conditions. CaNa₂EDTA is, for example, intravenously administered to treat lead poisoning.

Substances for use in the present invention are readily obtainable and are used as medications to treat other conditions. For example, CaNa EDTA is intravenously administered to treat lead poisoning. N-acetyl-cysteine is routinely used in many applications for example, for treatment of neurodegenerative diseases, chronic lung diseases and others. Dosing of NAC is described in G. C. Riise et al. (1994), Respir. J. 7:94-101, and in U.S. Pat. No. 4,331,648, (e.g., 2-150 mg of NAC per kilogram body weight) both are incorporated by reference as if fully set forth herein. Dosages for various collagenase inhibitors are described in the 1996 biochemicals catalog of Boehringer Mannheim, pages 460-465, which are incorporated by reference as if fully set forth herein.

Experimentation can be used to optimise the effective amount of the above substances required to be used in the pharmaceutical composition in accordance with the invention. The amount of these substances to give the desired result should be non-toxic to female mammals and the foetus. This may be effectively achieved by stimulating, enhancing or increasing the activity or amount of intravenously or application into the cervix or vaginal; or by any one or more such routes. One ordinarily skilled in the art would know how to devise a general or patient specific dosing program.

The pharmaceutical composition and method of the present invention may be administered in conjunction or in combination with other agents or methods which heretofore had been used in an endeavour to stop or prevent premature membranes rapture, cervical ripening and preterm labor. These include MgSO₄, beta mimetics, such as salbutamol, ritodrin and indomethacin, Ca⁺⁺blocker, oxytocin receptor antagonists, atosiban and antibiotics applied intravenously.

According to the present invention administration of the pharmaceutical composition may be administered in various forms and by various routes e.g., subcutaneously, intravenously, intramuscularly, orally, intracervically, intramniotically, extramniotically and intravaginally and therefore the pharmaceutical composition may by provided in the form of a liquid or solid formulation, including, but not limited to creme, ointment, gel, liquid, spray, powder, pill, capsule and patch. Depending on the specific application, the pharmaceutical composition may additionally include pharmaceutically acceptable excipients, such as, but not limited to, thickeners, carriers, buffers, diluents, surface active agents and preservatives, all as well known in the art of pharmacology.

While the invention has been described with respect to a limited number of embodiments, it will be appreciated that many variations, modifications and other applications of the invention may be made.

1. A method of treating or preventing premature ripening of the cervix, premature rupture of fetal membranes and preterm labor in a pregnant female comprising administering to the pregnant female:
N-acetyl-cysteine; and
a compound for reversing a high level of high affinity receptor mediated oxytocin effect;
thereby treating or preventing premature ripening of the cervix, premature rupture of fetal membranes and/or preterm labor in the pregnant female mammal.

2. The method of claim 1, wherein said compound for reversing said high level of high affinity receptor mediated oxytocin effect is selected from the group consisting of oxytocinase and an oxytocin receptor antagonist.

3. The method of claim 1, further comprising administering a conventional treatment for preventing ripening of the cervix and preterm labor.

4. The method of claim 3, wherein said conventional treatment is selected from the group consisting of treatment with MgSO₄, beta mimetic, Ca blocker, Atosiban and antibiotics.

5. The method of claim 4, wherein said beta mimetic is selected from the group consisting of salbutamol and ritodrin.

6. The method of claim 1, wherein said administration for said N-acetyl-cysteine and/or said compound is effected via a route selected from the group consisting of subcutaneously, intravenously, intramuscularly, orally, intracervically, intramniotically, extramniotically and intravaginally.

7. The method of claim 1, wherein said N-acetyl-cysteine and/or said compound are administered in a form selected from group consisting of cream, ointment, gel, liquid, spray, powder, pill, capsule and patch.

8. A pharmaceutical composition for treating or preventing premature ripening of the cervix, premature rupture of fetal membranes and/or preterm labor in a pregnant female pharmaceutical composition comprising, as active ingredients, N-acetyl-cysteine and a compound for reversing a high level of high affinity receptor mediated oxytocin effect, the pharmaceutical composition further comprising a pharmaceutically acceptable carrier.

9. The pharmaceutical composition of claim 8, wherein said compound for reversing a high level of high affinity receptor mediated oxytocin effect is selected from the group consisting of oxytocinase and an oxytocin receptor antagonist.

10. The pharmaceutical composition of claim 8, further comprising a compound used in conventional treatment for preventing ripening of the cervix and preterm labor.

11. The pharmaceutical composition of claim 10, wherein said compound used in conventional treatment is selected from the group consisting of MgSO₄, beta mimetic, Ca blocker, Atosiban and antibiotics.

12. The pharmaceutical composition of claim 10, wherein said beta mimetic is selected from the group consisting of salbutamol and ritodrin.

13. The pharmaceutical composition of claim 8, formulated for intravenous administration.

14. The pharmaceutical composition of claim 8, formulated as a cream, ointment, gel, liquid, spray, powder, pill, capsule or patch.

15. A method of treating or preventing premature ripening of the cervix, premature rupture of fetal membranes and preterm labor in a pregnant female comprising administering to the pregnant female:
an inhibitor of cervical collagenase; and
a compound for reversing a high level of high affinity receptor mediated oxytocin effect;
thereby treating or preventing premature ripening of the cervix, premature rupture of fetal membranes and/or preterm labor in the pregnant female mammal.

16. The method of claim 15, wherein said inhibitor of cervical collagenase is selected from a group consisting of caffeic acid, hydroxyquinoline, collagenase-inhibiting hydroxyquinoline derivative, phosphonepeptide, bencarbonyl-specified peptide sequence, a collagenase-inhibiting peptide sequence, anticollagenase antibodies, a collagenase-inhibiting tripeptide hydroxamic acid derivative, CaNa₂EDTA, alpha-2-macroglobulin, alpha-1-antitrypsin, a metalloprotease inhibitor, a cysteine proteinase inhibitor, N-acetyl-cysteine, N-acetylhomocysteine, N,N′-diacetylcysteine, L-arginine, guanido-substituted arginines or homoarginines, a collagenase inhibiting L-arginine N^{G} alkyl derivative, glycerol trinitrate and tissue inhibitor of matrix protease.

17. A pharmaceutical composition for treating or preventing premature ripening of the cervix, premature rupture of fetal membranes and/or preterm labor in a pregnant female pharmaceutical composition comprising, as active ingredients, an inhibitor of cervical collagenase and a compound for reversing a high level of high affinity receptor mediated oxytocin effect, the pharmaceutical composition further comprising a pharmaceutically acceptable carrier.

18. The pharmaceutical composition of claim 17, wherein said inhibitor of cervical collagenase is selected from a group consisting of caffeic acid, hydroxyquinoline, collagenase-inhibiting hydroxyquinoline derivative, phosphonepeptide, bencarbonyl-specified peptide sequence, a collagenase-inhibiting peptide sequence, anticollagenase antibodies, a collagenase-inhibiting tripeptide hydroxamic acid derivative, CaNa₂EDTA, alpha-2-macroglobulin, alpha-1-antitrypsin, a metalloprotease inhibitor, a cysteine proteinase inhibitor, N-acetyl-cysteine, N-acetylhomocysteine, N,N′-diacetylcysteine, L-arginine, guanido-substituted arginines or homoarginines, a collagenase inhibiting L-arginine N^{G} alkyl derivative, glycerol trinitrate and tissue inhibitor of matrix protease.
